(19) United States
(12) Patent Application Publication
(10) Pub. No.: US 2014/0358174 A1
(43) Pub. Date: Dec. 4, 2014

(54) Intraneural Implant

(71) Applicants: Chuladatta Thenuwara, Castaic, CA (US); Mark B. Downing, Valencia, CA (US)

(72) Inventors: Chuladatta Thenuwara, Castaic, CA (US); Mark B. Downing, Valencia, CA (US)

(21) Appl. No.: 14/372,380

(22) PCT Filed: Jan. 25, 2012
(86) PCT No.: PCT/US2012/022531

§ 371 Date: Jul. 15, 2014

(51) Int. Cl. A61N 1/05

(52) U.S. Cl. CPC A61N 1/0541; A61B 2017/320044
USPC 606/190; 607/137

(57) ABSTRACT

An intraneural implant comprises a lead comprising a number of electrode wires, a number of electrodes communicatively coupled to the electrode wires, the electrodes forming an electrode array, and an overmold surrounding the electrode wires and at least a portion of the electrodes, and a blunt dissector tip coupled to the lead to penetrate nerve tissues as the electrode array is implanted. An intraneural implant system comprises a flexible lead. The flexible lead comprises a lead body, an electrode array communicatively coupled to the lead body, a blunt dissector tip to penetrate a nerve bundle as the electrode array is implanted into the nerve bundle, and an implantation tool coupled to the electrode array to implant the electrode array into the nerve bundle.

### BACKGROUND

In human hearing, hair cells in the cochlea respond to sound waves and produce corresponding cochlear nerve impulses. These nerve impulses are then conducted via the cochlear nerve to the brain and perceived as sound. Hearing loss, which may be due to many different causes, is generally of two types: conductive and sensorineural. Conductive hearing loss typically occurs where the normal mechanical pathways for sound to reach the hair cells in the cochlea are impeded, for example, from damage to the ossicles. Conductive hearing loss may often be helped by using hearing aids that amplify sounds so that acoustic information can reach the cochlea and the hair cells. Some types of conductive hearing loss are also treatable by surgical procedures.

Many people who are profoundly deaf, however, have sensorineural hearing loss. This type of hearing loss can arise from the absence or the destruction of the hair cells in the cochlea that then no longer transduce acoustic signals into cochlear nerve impulses. Individuals with sensorineural hearing loss may be unable to derive significant benefit from hearing aid systems alone, no matter how loud the acoustic stimulus is. This is because the natural mechanism for transducing sound energy into cochlear nerve impulses has been damaged. Thus, in the absence of properly functioning hair cells, cochlear nerve impulses cannot be generated directly from sounds.

To overcome sensorineural deafness, cochlear implant systems have been developed that can bypass the hair cells located in the cochlea by presenting electrical stimulation to the cochlear nerve fibers via the cochlear pathways. This leads to the perception of sound in the brain and provides at least partial restoration of hearing function. Some cochlear implant systems treat sensorineural deficit by stimulating the ganglion cells in the cochlea directly using an implanted electrode or lead that has an intraneural electrode array. Thus, a cochlear implant operates by stimulating the cochlear nerve cells via the cochlea, bypassing the defective cochlear hair cells that normally transduce acoustic energy into electrical activity in the connected cochlear nerve cells. However, in cases where the scala tympani has been fully or severely obstructed, such as in some severe cases of ossification of the cochlea, implantation of a cochlear implant in the cochlea is not a viable solution to sensorineural deafness.

### BRIEF DESCRIPTION OF THE DRAWINGS

The accompanying drawings illustrate various examples of the principles described herein and are a part of the specification. The illustrated examples are given merely for illustration, and do not limit the scope of the claims.

FIG. 1 is a diagram of an intraneural implant system in use, according to one example of principles described herein.

FIG. 2 is a diagram of the external components of the intraneural implant system of FIG. 1, according to one example of principles described herein.

FIG. 3 is a diagram of the internal components of the intraneural implant system of FIG. 1, according to one example of principles described herein.

FIG. 4 is a cross-sectional view of a human cochlea and cochlear nerve with an intraneural lead comprising an intraneural electrode array coupled to the cochlear nerve, according to one example of principles described herein.

FIG. 5 is a perspective view of an intraneural electrode array implantation system, according to one example of the principles described herein.

FIG. 6 is a perspective view of the distal end of an intraneural lead of the intraneural electrode array implantation system of FIG. 5, according to one example of the principles described herein.

FIG. 7 is a perspective view of section A of the intraneural lead of FIG. 6, according to one example of the principles described herein.

FIG. 8 is a perspective view of the distal end of an intraneural lead of the intraneural electrode array implantation system of FIG. 5, according to another example of the principles described herein.

FIG. 9 is a perspective view of an intraneural lead of the intraneural electrode array implantation system of FIG. 5, according to still another example of the principles described herein.

FIG. 10 is a perspective view of an intraneural electrode array implantation system, according to another example of the principles described herein.

FIG. 11 is a perspective view of the distal end of an intraneural lead of the intraneural electrode array implantation system of FIG. 10, according to one example of the principles described herein.

FIG. 12 is a perspective view of the implantation tool of the intraneural electrode array implantation system of FIG. 10, according to one example of the principles described herein.

FIG. 13 is an exploded perspective view of an intraneural electrode array implantation system, according to still another example of the principles described herein.

FIG. 14 is a perspective view of an intraneural lead, according to yet another example of the principles described herein.

FIG. 15 is a perspective view of the stiffener of the intraneural lead of FIG. 14, according to one example of the principles described herein.

FIG. 16 is a perspective view of an intraneural lead, according to yet another example of the principles described herein.

FIG. 17 is a perspective view of the stiffener of the intraneural lead of FIG. 16, according to one example of the principles described herein.

FIG. 18 is a perspective view of an intraneural lead comprising circular ring electrodes, according to yet another example of the principles described herein.

FIG. 19 is a perspective view of an intraneural lead comprising elliptical ring electrodes, according to yet another example of the principles described herein.

Throughout the drawings, identical reference numbers designate similar, but not necessarily identical, elements.

### DETAILED DESCRIPTION

The implantation of a cochlear implant system involves the insertion of an intraneural electrode array into the cochlea of the patient. The present systems and methods describe an intraneural implant implantable into nerve tissues, and, more specifically, into cochlear nerve tissues. Thus, the present intraneural implant bypasses the ganglion cells of the cochlea of the inner ear and directly stimulates the cochlear nerve. Further, the intraneural implant stimulates the cochlear nerve in a more selective manner than a cochlear implant. Further, the intraneural implant stimulates the cochlear nerve at lower thresholds and with greater frequency selectivity than a cochlear implant.

Intraneural implants comprising rigid materials may be used to directly stimulate the spiral ganglion and/or the cochlear nerve. However, many intraneural implants expose a user to the possibility of nerve damage produced by movement of the cochlear nerve relative to rigid materials and the lack of flexible wires to stimulate an implanted electrode. Further, implantation of an intraneural implant into the cochlear nerve may result in damage to the cochlear nerve as the elements that come into contact with the cochlear nerve may break or cut a number of nerve fibers. Still further, intraneural implants comprising interconnects between an electrode array and a lead body may not be reliable.

The present systems and methods describe an intraneural implant comprising a lead. The lead comprises a number of electrode wires, and a number of electrodes communicatively coupled to the electrode wires. The number of electrodes may form an intraneural electrode array. The intraneural implant further comprises an overmold surrounding the lead. A blunt dissector tip may be used with or coupled to the intraneural implant to penetrate nerve tissues as the intraneural electrode array of the intraneural implant is implanted in the cochlear nerve. Further, the present systems and methods describe an intraneural implant system comprising an implantation tool coupled to the lead to implant the intraneural electrode array into the nerve bundle.

The present systems and methods allow for direct stimulation of the cochlear nerve via the intraneural implant in situations where a cochlear implant is ineffective or in situations where direct stimulation of the cochlear nerve via the intraneural implant is used in concert with a cochlear implant. Thus, in one example, the present intraneural implant may be employed singularly or in connection with a cochlear implant system.

As used in the present specification and in the appended claims, the term “intraneural array” is meant to be understood broadly as any array situated within a nerve or nervous tissue of an organism, or any array that is implanted or implantable into a nerve or nervous tissue of an organism. In one example, an intraneural array is any array that is implanted or implantable into a cochlear nerve of a human.

As used in the present specification and in the appended claims, the term “bodily tissues” is meant to be understood broadly as any organic tissues. In one example, bodily tissues may include living and cadaver tissues. In another example, bodily tissues may include human tissues. In still another example, bodily tissues may include tissues through which an intraneural electrode array implantation system is navigated to implant an intraneural electrode or intraneural electrode array into a cochlear nerve. In yet another example, bodily tissues may include tissues of a species of organism that has a cochlear nerve. In still another example, bodily tissues may include tissues of organisms other than homo sapiens.

Further, as used in the present specification and in the appended claims, the terms “coupled,” “selectively coupled,” “selectively engaged,” “selectively engage or disengage,” or similar language is meant to be understood broadly as any first element that is connected to and disconnected from a corresponding second element within an associated device without disassembly or destruction of either the first or second elements.

Further, as used in the present specification and in the appended claims, the term “a number of” or similar language is meant to be understood broadly as any positive number comprising 1 to infinity; zero not being a number, but the absence of a number.

In the following description, for purposes of explanation, numerous specific details are set forth in order to provide a thorough understanding of the present systems and methods. It will be apparent, however, to one skilled in the art that the present apparatus, systems, and methods may be practiced without these specific details. Reference in the specification to “an example” or similar language means that a particular feature, structure, or characteristic described in connection with that example is included as described, but may not be included in other examples.

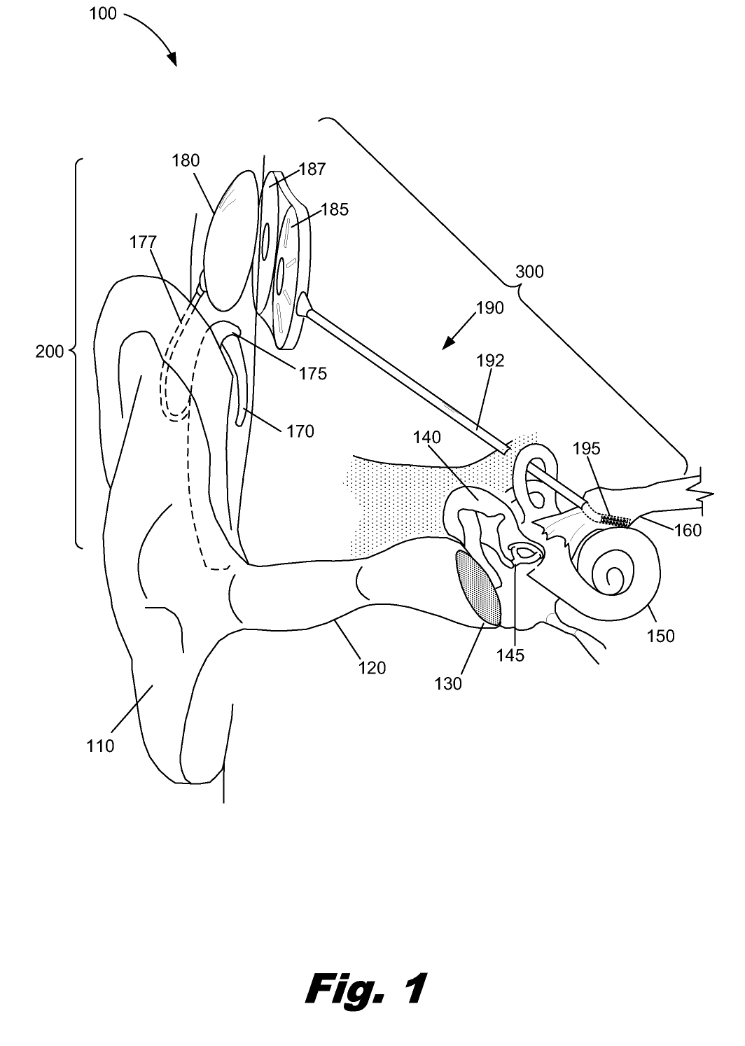

FIG. 1 is a diagram of an intraneural implant system (100) in use, according to one example of principles described herein. The intraneural implant system (100) comprises an intraneural implant (300) comprising an intraneural electrode lead (190). The intraneural electrode lead (190) comprises a lead body (192) and an intraneural electrode array (195) that is surgically positioned adjacent to the patient's cochlea (150) and in contact with or inserted into the patient's cochlear nerve (160). Ordinarily, sound enters the external ear, or pinna (110), and is directed into the auditory canal (120) where the sound wave vibrates the tympanic membrane (130). The motion of the tympanic membrane (130) is amplified and transmitted through the ossicular chain (140), which includes three bones in the middle ear. The third bone of the ossicular chain (140), the stapes (145), contacts the outer surface of the cochlea (150) and causes movement of the fluid within the cochlea (150). Cochlear hair cells respond to the fluid-borne vibration in the cochlea (150) and trigger neural electrical signals that are conducted from the cochlea (150) to the auditory cortex of the brain via the cochlear nerve (160).

As indicated above, the intraneural implant (300) comprises a surgically implanted electronic device that provides a sense of sound to a person who is profoundly deaf or severely hard of hearing. The intraneural implant (300) operates by direct electrical stimulation of the cochlear nerve cells, bypassing the cochlea, spiral ganglion neurons, and the cochlea's defective hair cells that normally transduce acoustic energy into electrical energy.

In one example, the intraneural implant (300) is utilized in conjunction with a cochlear implant comprising an intraneural electrode array implanted into the cochlea. In this example, a number of intraneural implants (300) and a number of cochlear implants may be introduced into their respective portions of the inner ear. In this example, the intraneural implants (300) and cochlear implants are utilized in a synergistic manner to improve selectivity and to stimulate low-frequency fibers located at the core of the cochlear nerve. Further, the example incorporating the intraneural implants (300) and cochlear implants provide loudness growth through intra-cochlear stimulation and act as a reference array for the programming of an intra-cochlear electrode array.

External components (200) of the intraneural implant system (100) comprise a Behind-The-Ear (BTE) unit (175) or similar unit, which contains a sound processor and further comprises a microphone (170), a cable (177), and a transmitter (180). The microphone (170) picks up sound from the environment and converts it into electrical impulses. The sound processor within the BTE unit (175) selectively filters and manipulates the electrical impulses and sends the processed electrical signals through the cable (177) to the transmitter (180). The transmitter (180) receives the processed electrical signals from the processor and transmits them to the implanted antenna (187) by electromagnetic transmission.

The components of the intraneural implant (300) include an internal processor (185), an antenna (187), and an intraneural electrode lead (190) comprising a lead body (192) and an intraneural electrode array (195). In one example, the internal processor (185) and antenna (187) are secured beneath the user's skin above and behind the pinna (110). The antenna (187) receives signals and power from the transmitter (180). The internal processor (185) receives these signals and performs a number of operations on the signals to generate modified signals. These modified signals are then sent along a number of signal wires that pass through the intraneural electrode lead (190) and are individually connected to the electrodes in the intraneural electrode array (195). The intraneural electrode array (195) is brought into contact with or is implanted within the cochlear nerve (160) and provides electrical stimulation to the cochlear nerve (160).

The intraneural implant (300) stimulates different portions of the cochlear nerve (160) according to the frequencies detected by the microphone (170). This is similar to how a normal functioning ear would experience stimulation at different portions of the cochlea and respective cochlear nerve tissues depending on the frequency of sound vibrating the endolymph within the scala media of the cochlea (150). This allows the brain to interpret the frequency of the sound as if the hair cells of the basilar membrane within the cochlea (150) were functioning properly.

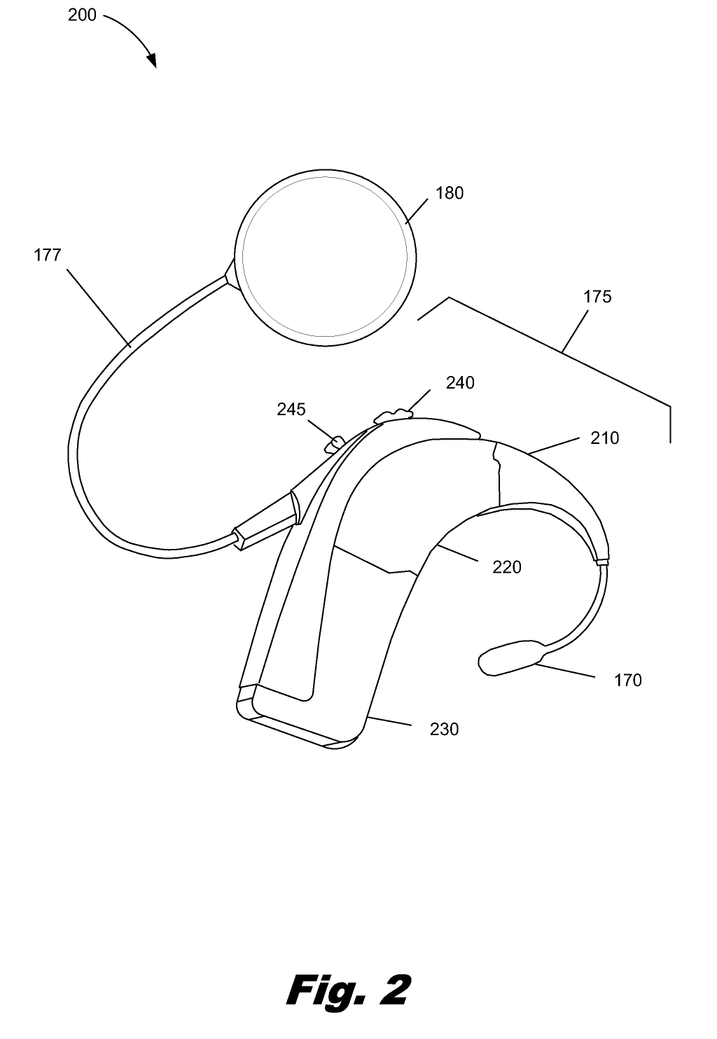

FIG. 2 is a diagram of the external components (200) of the intraneural implant system (100) of FIG. 1, according to one example of principles described herein. External components (200) of the intraneural implant system (100) include a behind-the-ear (BTE) unit (175), which comprises a microphone (170), an ear hook (210), a sound processor (220), and a battery (230). In one example, the battery (230) may be rechargeable. The microphone (170) picks up sound from the environment and converts it into electrical impulses. The sound processor (220) selectively filters and manipulates the electrical impulses and sends the processed electrical signals through a cable (177) to the transmitter (180). A number of controls (240, 245) adjust the operation of the processor (220). These controls may include a volume switch (240) and program selection switch (245). The transmitter (180) receives the processed electrical signals from the processor (220) and transmits these electrical signals and power from the battery (230) to the intraneural implant (300) by electromagnetic transmission.

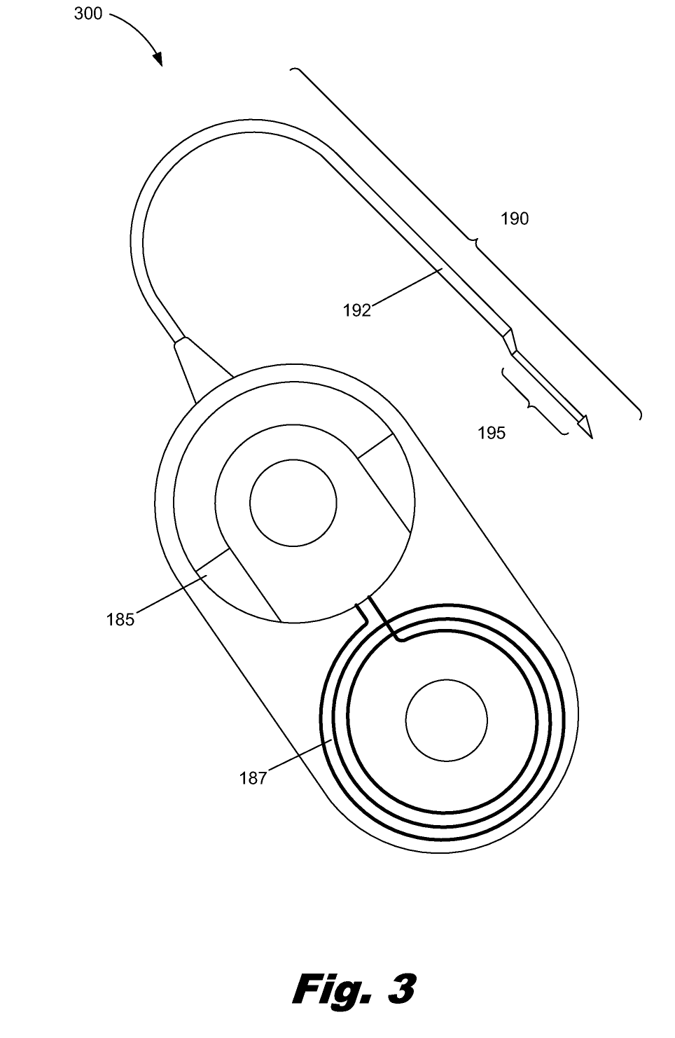

FIG. 3 is a diagram of the internal components of the intraneural implant system (100) of FIG. 1, according to one example of principles described herein. The internal components comprise the intraneural implant (300) of the intraneural implant system (100). The intraneural implant (300) comprises an internal processor (185), an antenna (187), and an intraneural electrode lead (190) comprising a lead body (192) and an intraneural electrode array (195). The intraneural implant (300) is surgically implanted such that the intraneural electrode array (195) is brought into contact with or implanted within the cochlear nerve (160), as shown in FIG. 1.

The internal processor (185) and antenna (187) are secured beneath the user's skin above and behind the pinna (110), with the intraneural electrode lead (190) connecting the internal processor (185) to the intraneural electrode array (195). As discussed above, the antenna (187) receives signals from the transmitter (180) and sends the signals to the internal processor (185). The internal processor (185) modifies the signals and passes them along the appropriate wires to activate a number of the electrodes within the intraneural electrode array (195). This provides the user with sensory input that is a representation of external sound waves sensed by the microphone (170).

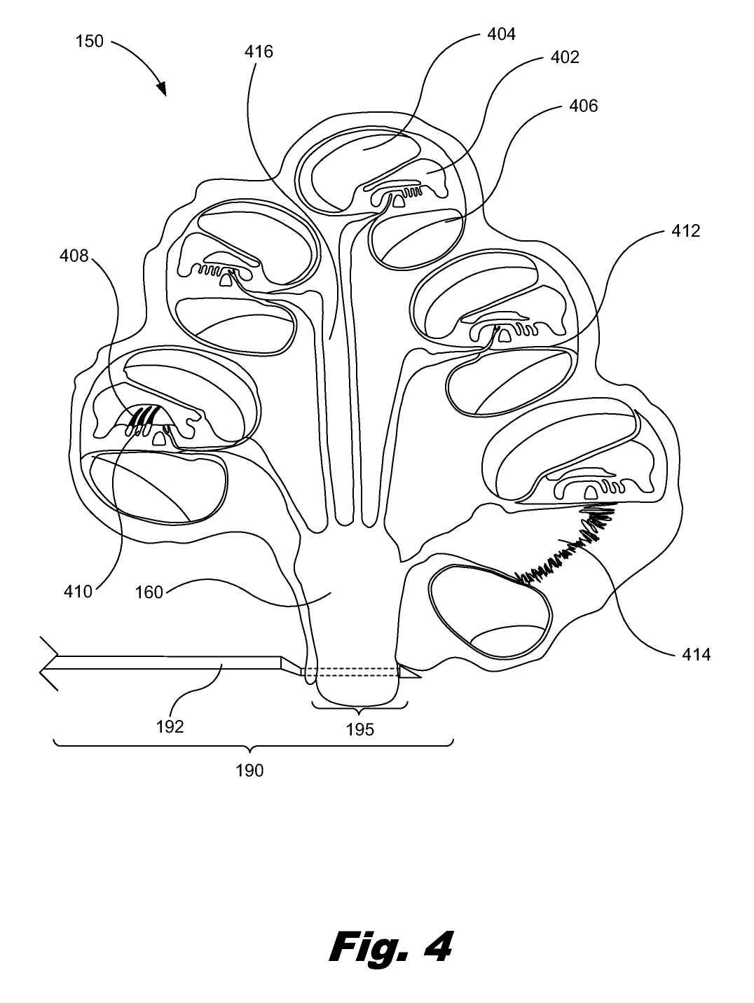

FIG. 4 is a cross-sectional view of a human cochlea (150) and cochlear nerve (160) with an intraneural electrode lead (190) comprising a lead body (192) and an intraneural electrode array (195) coupled to the cochlear nerve (160), according to one example of principles described herein. The cochlea (150) comprises three scalae (402, 404, 406). The scala media (402) into which the cilia (408) of the hair cells (410) project is located between the scala vestibuli (404) and the scala tympani (406). The cilia (408) of the hair cells (410) project from the basilar membrane (412) and produce electrical signals that are then relayed via the cochlear nerve (160) to the auditory brainstem and to the auditory cortex.

The spiral ganglion neurons (414) that electrically couple the cilia (408) of the hair cells (410) to the cochlear nerve (160) reach from the hair cells (410) to the cochlear nerve (160), and are generally located in the modiolus (416). The intraneural electrode array (195) of the intraneural electrode lead (190) may be positioned within the cochlear nerve at any point at which tonotopic excitation may be registered and relayed to the auditory brainstem and to the auditory cortex. In one example, the intraneural electrode array (195) is positioned within the bundle of nerve fibers that comprise the cochlear nerve (160). In this example, the intraneural electrode array (195) may be inserted via a blunt dissector tip that enters the bundle of nerve fibers without cutting, as will be described in more detail below.

In another example, the intraneural electrode array (195) is juxtaposed to an area where the spiral ganglion neurons (414) are first brought together in a group at the top of the bundle of nerve fibers that comprise the cochlear nerve (160). In this example, the intraneural electrode array (195) may be positioned within the cochlear nerve (160) relatively closer to the portion where the individual spiral ganglion neurons (414) draw together to form the cochlear nerve (160). This example of intraneural electrode array (195) insertion may provide for more localized excitation of nerve fibers that register specific tones.

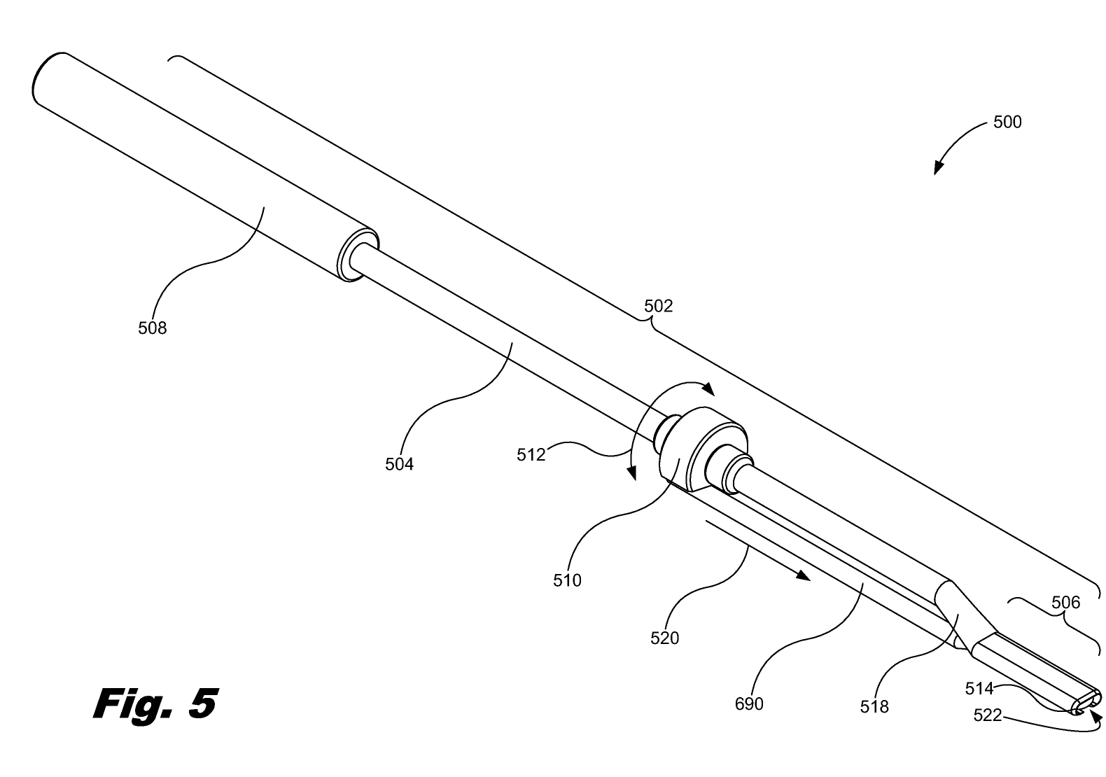

FIG. 5 is a perspective view of an intraneural electrode array implantation system (500), according to one example of the principles described herein. The implantation system (500) comprises an implantation tool (502) and an intraneural electrode lead (690) selectively coupled to the implantation tool (502). The implantation tool (502) comprises a main shaft (504), a handle (508) coupled to the main shaft (504), and a lead receiving portion (506). The handle (508) is used by, for example, a physician in steering the implantation system (500) during implantation of the intraneural electrode lead (690). The main shaft (504), handle (508), and lead receiving portion (506) of the implantation tool (502) are made of generally rigid and durable material so that the implantation tool (502) can enter bodily tissues without diverging from an intended insertion path or breaking due to stress. In one example, the implantation tool (502) is made of stainless surgical steel.

Use of an implantation tool (502) in this example and other examples described herein assists in the elimination or reduction of infection that may occur. This is because the implantation tool (502) of this and other examples described herein does not include an open lumen in which pathogens may enter into the organism. During implantation of the intraneural electrode lead (690) of this example and others described herein, access to the cochlear nerve (160) is achieved via the middle ear. The middle ear comprises a hollow space called the tympanic cavity. The Eustachian tube joins the tympanic cavity with the nasal cavity, allowing pressure to equalize between the middle ear and throat. However, pathogens may travel up the Eustachian tube to the middle ear. The implantation of a lumen structure through the middle ear and into the inner ear where the cochlear nerve (160) is located may result in infection. Thus, the closed environment due to the lack of any type of lumen within the intraneural electrode lead (690), and the lack of an open lumen within the implantation tool (502) of this and other examples described herein eliminates or reduces the chance of infection within the cochlear nerve (160) or inner ear.

In one example, a locking ring (510) may be incorporated into the main shaft (504) of the implantation tool (502). The locking ring (510) may be turned as indicated by arrow (512) to selectively engage or disengage the intraneural electrode lead (690) with or from the lead receiving portion (506) of the implantation tool (502) of the implantation system (500). The locking ring (510) allows for the intraneural electrode lead (690) to remain engaged with the implantation tool (502) during implantation of the intraneural electrode lead (190) when the implantation tool (502) is pushed through various bodily tissues and into the cochlear nerve (160). Further, the locking ring (510) allows for the intraneural electrode lead (690) to separate from the implantation tool (502) when the intraneural electrode lead (690) is positioned within the cochlear nerve (160) and the implantation tool (502) is removed.

In one example, the distal end of the lead receiving portion (506) of the implantation tool (502) comprises a blunt dissector tip (514) that enables the implantation tool (502) to move through bodily tissues without cutting the tissues. For example, when the implantation tool (502) is inserted into nerve tissues such as the cochlear nerve (160), the blunt dissector tip (514) allows the implantation tool (502) to separate and move past nerve tissues. Thus, the blunt dissector tip (514) of the implantation tool (502) eliminates or reduces the cutting of nerve cells.

In the example of FIG. 5, the lead receiving portion (506) comprises an opening (522) along the side of the lead receiving portion (506). In the example of FIG. 5, the lead receiving portion (506) comprises a generally c-shaped cross section with respect to the longitudinal axis of the implantation tool (502). In another example, the lead receiving portion (506) may, instead, have a square, circular, elliptical, or polygonal cross section. A number of alternative examples of the cross section of the lead receiving portion (506) will be described below in connection with other examples.

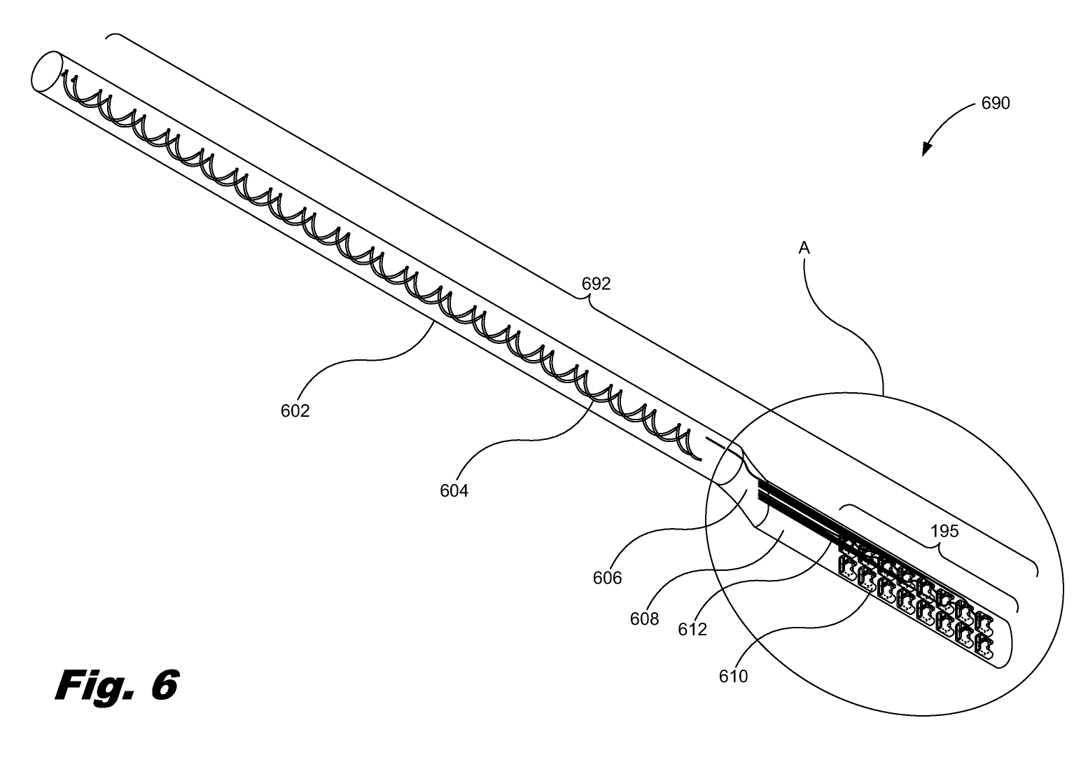

An electrode substrate portion (FIG. 6, 608) of the intraneural electrode lead (FIG. 6, 690) may have a cross section (FIG. 6, 614) so that the intraneural electrode lead (FIG. 6, 690) couples with the implantation tool (502) in a nesting manner. Thus, in this example and other examples described herein, the electrode substrate portion (FIG. 6, 608) of the intraneural electrode lead (FIG. 6, 690) may be formed such that an interference fit exists between the lead receiving portion (506) and the electrode substrate portion (FIG. 6, 608) of the intraneural electrode lead (FIG. 6, 690).

The implantation tool (502) may further comprise a diverging arm (518) that angularly extends from the main shaft (504) to the lead receiving portion (506). The diverging arm (518) axially separates the main shaft (504) from the lead receiving portion (506). By axially separating the main shaft (504) from the lead receiving portion (506), the intraneural lead (690), when coupled to the implantation tool (502), remains behind the lead receiving portion (506) and below the main shaft (504) and is not displaced or disengaged from the implantation tool (502) when the implantation tool (502) is inserted into bodily tissues.

Moving to the next figure, FIG. 6 is a perspective view of the distal end of an intraneural electrode lead (690) of the intraneural electrode array implantation system (500) of FIG. 5, according to one example of the principles described herein. The intraneural electrode lead (690) comprises a lead body (692) comprising a main lead portion (602), a lead diverging arm (606), and an electrode substrate portion (608). Although depicted as a truncated element in FIG. 6, the main lead portion (602) extends from the internal processor (FIG. 1, 185) of the intraneural implant (FIG. 1, 300) to the cochlear nerve (FIG. 1, 160).

The lead diverging arm (606) angularly extends from the main lead portion (602) to the electrode substrate portion (608). In one example, the contour formed by the main lead portion (602), lead diverging arm (606), and electrode substrate portion (608) matches the contour formed by the main shaft (FIG. 5, 504), diverging arm (FIG. 5, 518), and lead receiving portion (FIG. 5, 506) as depicted in FIG. 5.

The electrode substrate portion (608) provides a portion of the intraneural electrode lead (690) to which the electrodes (610) of the intraneural electrode array (195) is coupled. As described above, the intraneural electrode lead (690) comprises an intraneural electrode array (195) comprising a number of electrodes (610) that are electronically coupled to the internal processor (FIG. 1, 185) of the intraneural implant (FIG. 1, 300) via a number of electrode wires (612). In one example, an electrode wire (612) extends from each electrode (610).

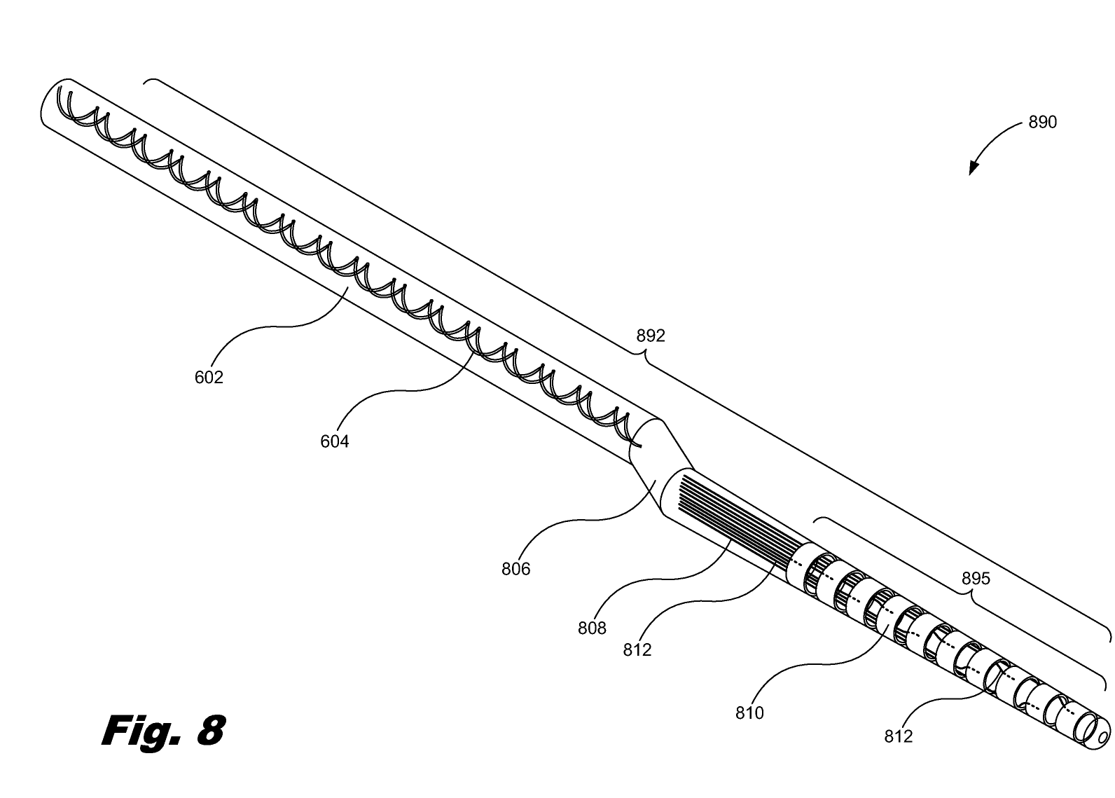

The electrode wires (612) for only one bank of electrodes (610) are depicted here in FIG. 6, in order to show details within the electrodes (610). However, in this example and each example described herein, an electrode wire (612) extends from each electrode (610). Further, in this and other examples described herein, electrode wires (612) extend from the electrodes to the coiled electrode wires (604). However, in several examples described herein, the electrode wires are omitted from the drawings to show arrangement of electrodes and other elements, to show detail within the figures, and to simplify the drawings. Thus, other examples described herein may be analogized to, for example, FIGS. 6 and 8 in determining the arrangement of electrode wires within the several examples.

The electrode wires (612) aggregate into a number of coiled electrode wires (604). The coiled electrode wires (604) comprise the individual and electrically isolated electrode wires (612) grouped together side-by-side in a bundle. In the example of FIG. 6, two coiled electrode wires (604) are depicted. However, a number of electrode wires (612) may be bundled to form any number of coiled electrode wires (604). The coiled electrode wires (604), in this example and other examples described herein, act as strain relief loops. If a portion of the intraneural implant (FIG. 3, 300) such as, for example, the internal processor (FIG. 1, 185) or the intraneural electrode lead (690) should be dislodged or moved from an original implantation position within the bodily tissues through, for example, a force applied to the bodily tissues surrounding the implantation area, the coiled electrode wires (604) dampen the movement with respect to the intraneural electrode array (195). In this manner, the risk of dislodgement or movement of the intraneural electrode array (195) with respect to an originally implanted position within the cochlear nerve (160) is reduced or eliminated.

In one example, the coiled electrode wires (604) and electrode wires (612) are made of platinum (Pt) or a platinum alloy such as, for example, a platinum-iridium (Pt—Ir) alloy. In one example, the electrode wires (612) are coupled to their respective electrodes (610) by welding, laser welding, resistance welding, or combinations thereof.

The intraneural electrode lead (690) of the example of FIG. 6 and other examples described herein is made of a flexible material. An advantage of making the intraneural lead out of a flexible material is to prevent the rigid material from becoming dislodged from its originally implanted position and to allow the intraneural electrode lead (690) to move within the tissues in which it is implanted. Flexibility in the intraneural electrode lead (690) reduces or eliminates movement of the intraneural electrode lead (690) with respect to bodily tissues including the cochlear nerve (160).

Further, organic tissues often tend to encapsulate in fibrous tissues rigid objects implanted into the organic tissues and expel the rigid objects. Thus, for the above reasons, a flexible intraneural electrode lead (690) may remain in the bodily tissues without moving from an originally implanted position and without being attacked or expelled from the bodily tissues in which it is implanted.

In the example of FIG. 6 and other examples described herein, the intraneural electrode lead (690) is made of a polysiloxane, such as medical grade silicone. In one example, the intraneural electrode lead (690) is made of a material with a Shore A hardness of between approximately 30 and 70 durometer. In this example and other examples described herein, the electrodes (610) and/or intraneural electrode arrays (195), electrode wires (612), and coiled electrode wires (604) are at least partially overmolded with silicone. Portions of the electrodes (610) and/or intraneural electrode array (195) are exposed to bodily tissues to electrically stimulate a portion of the cochlear nerve (160) into which the electrodes (610) and/or intraneural electrode arrays (195) are inserted. Use of a silicone overmold in this example and other examples described herein assist in the elimination or reduction of infection that may occur if one or more portions of the intraneural electrode lead (690) were exposed to bodily tissues. The intraneural electrode lead (690), with its silicone overmold, does not comprise cavities or lumen in which pathogens may enter and be exposed to bodily tissues. Thus, the closed environment of the intraneural electrode lead (690) due to the silicone overmolding eliminates or reduces the chance of infection within the cochlear nerve (160).

The electrodes (610) and/or intraneural electrode arrays (195) of the example of FIG. 6 and other examples described herein are made of a material that can withstand an in vivo environment. In one example, the electrodes (610) and/or intraneural electrode arrays (195) are made of platinum or a platinum alloy such as, for example, a platinum-iridium alloy.

The electrode substrate portion (608) of the intraneural electrode lead (690) is coupled to the lead receiving portion (FIG. 5, 506) of the implantation tool (FIG. 5, 502) by moving the electrode substrate portion (608) into the recess defined by the lead receiving portion (FIG. 5, 506) in the direction of arrow (520). After electrode substrate portion (608) is inserted into the recess defined by the lead receiving portion (FIG. 5, 506), the locking ring (510) is engaged to secure the intraneural electrode lead (190) to the implantation tool (502).

The electrode array (195) of FIG. 6 and other examples described herein may comprise any number of electrodes (610) arranged in any manner along the electrode substrate portion (608). In the example of FIG. 6, the intraneural electrode array (195) comprises a number of electrodes (610) that will excite a number of portions of the cochlear nerve (160). Stimulation of the portions of the cochlear nerve (160) in this manner produce signals to the brain via the cochlear nerve (160) that are tonotopically distinguishable by, for example, a patient. In one example, the intraneural electrode array (195) comprises between approximately 10 and 20 electrodes (610). In another example, the intraneural electrode array (195) comprises 16 electrodes (610) arranged on the electrode substrate portion (608) in eight parallel pairs as depicted in FIG. 6. In another example, the intraneural electrode array (195) comprises a number of electrodes (610) arranged in a single row. In still other examples, the electrodes (610) comprise rings, half rings, flat pads, multiple pins, multiple pads, or combinations thereof.

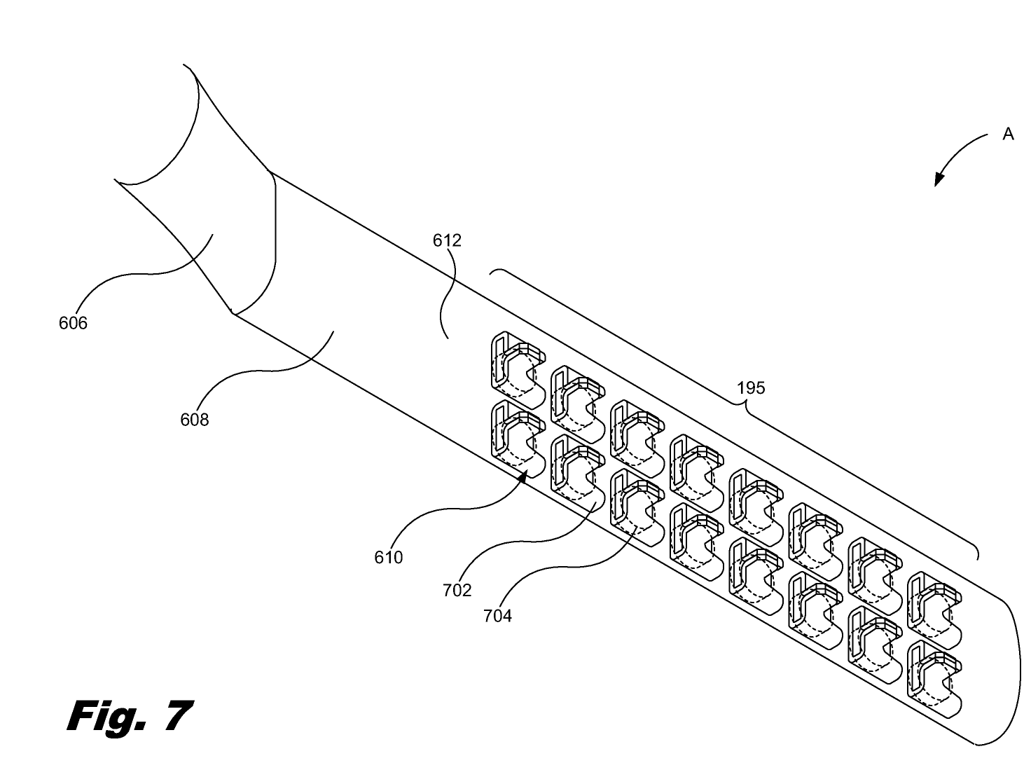

FIG. 7 is a perspective view of section A of the intraneural lead of FIG. 6, according to one example of the principles described herein. In the example depicted in section A of FIG. 7 and other examples described herein, the electrodes (610) may each comprise an electrode body (702) and a conductive pad (704). The conductive pad (704) of this and other examples described herein are shown in ghost using dashed lines. This is because the conductive pad (704) is electrically and mechanically coupled to the electrode body (702), and exposed to bodily tissues through the silicone overmold.

The electrode bodies (702) and conductive pads (704) are made of platinum or a platinum alloy such as, for example, a platinum-iridium alloy. Once implanted into a cochlear nerve (160), electrical signals are selectively passed from the internal processor (185), through the number of electrode wires (612) to the electrode bodies (702) and conductive pads (704) of the individual electrodes (610) to be activated. In this manner, the electrodes (610) stimulate the cochlear nerve (160), and provide the user with sensory input that is a representation of sound as described above.

FIG. 8 is a perspective view of the distal end of an intraneural electrode lead (890) of the intraneural electrode array implantation system (500) of FIG. 5, according to another example of the principles described herein. The intraneural electrode lead (890) comprises a lead body (892) comprising a main lead portion (602), a lead diverging arm (806), and an electrode substrate portion (808). Although depicted as a truncated element in FIG. 8, the main lead portion (602) extends from the internal processor (185) of the intraneural implant (300) to the cochlear nerve (160). In the example of FIG. 8, the lead diverging arm (806) and electrode substrate portion (808) have an approximately circular cross section.

The lead diverging arm (806) angularly extends from the main lead portion (602) to the electrode substrate portion (808). In one example, the contour formed by the main lead portion (602), lead diverging arm (806), and electrode substrate portion (808) matches the contour formed by the main shaft (FIG. 5, 504), diverging arm (FIG. 5, 518) and lead receiving portion (FIG. 5, 506) as depicted in FIG. 5.

The electrode substrate portion (808) provides a portion of the intraneural electrode lead (890) to which an intraneural electrode array (895) is coupled. The intraneural electrode lead (890) thus comprises a number of electrodes (810) or intraneural electrode arrays (895) that are electronically coupled to the internal processor (185) of the intraneural implant (300) via a number of connecting wires. In the example of FIG. 8, the electrodes (810) of the intraneural electrode array (895) are ring electrodes with a generally circular cross section. Similarly, the electrode substrate portion (808) also comprises a matching generally circular cross section. In another example of FIG. 8, the lead diverging arm (806), electrode substrate portion (808), and electrodes (808) have an elliptical cross section forming an intraneural electrode lead (890) with a cross section that has a major and minor axis.

In one example, an electrode wire (812) extends from each electrode (810). The electrode wires (812) are then aggregated into a number of coiled electrode wires (604); the coiled electrode wires (604) comprising the individual and electrically isolated electrode wires (812) grouped together side-by-side in a bundle. In the example of FIG. 8, two coiled electrode wires (604) are depicted. However, a number of electrode wires (812) may be bundled to form any number of coiled electrode wires (604).

Further, in conjunction with the example of FIG. 8, the lead receiving portion (506) of the implantation tool (FIG. 5, 502) may comprise a generally circular cross section with respect to the longitudinal axis of the implantation tool (FIG. 5, 502). The electrode substrate portion (808) and electrodes (810) mate with, and couple to the implantation tool (FIG. 5, 502). In this manner, the electrode substrate portion (808) of the intraneural electrode lead (890) couples with the implantation tool (FIG. 5, 502) in a nesting manner. Thus, the electrode substrate portion (FIG. 6, 608) of the intraneural electrode lead (890) may be formed such that an interference fit exists between the lead receiving portion (FIG. 5, 506) and the electrode substrate portion (808) of the intraneural electrode lead (890).

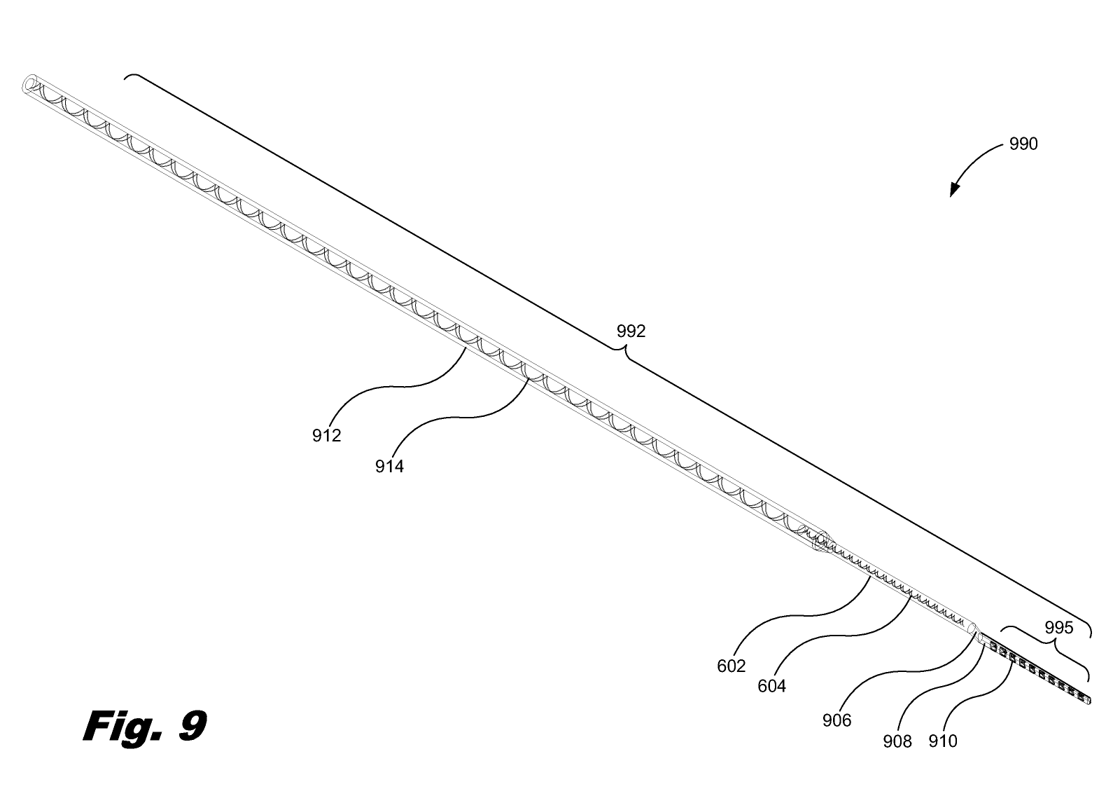

FIG. 9 is a perspective view of an intraneural electrode lead (990) of the intraneural electrode array implantation system (500) of FIG. 5, according to still another example of the principles described herein. The example intraneural electrode lead (990) depicted in FIG. 9 comprises a lead body (992) comprising a main lead portion (602) comprising a number of coiled electrode wires (604), a lead diverging arm (906), and an electrode substrate portion (608) comprising an intraneural electrode array (995) comprising a number of electrodes (910). The intraneural electrode array (995) of FIG. 9 comprises a single row of electrodes (910) like those utilized in the examples of FIGS. 6 and 7, and comprise an electrode body (702) and a conductive pad (704) as described above.

The example of FIG. 9 further comprises a proximal lead portion (912). The proximal lead portion (912) comprises a number of relatively larger coiled electrode wires (914). In this example, the coiled electrode wires (604) of the main lead portion (602) are formed into the larger coiled electrode wires (914) when the intraneural electrode lead (990) transitions from the main lead portion (602) to the proximal lead portion (912). In this manner, the intraneural electrode lead (990) is further extended and is made more robust.

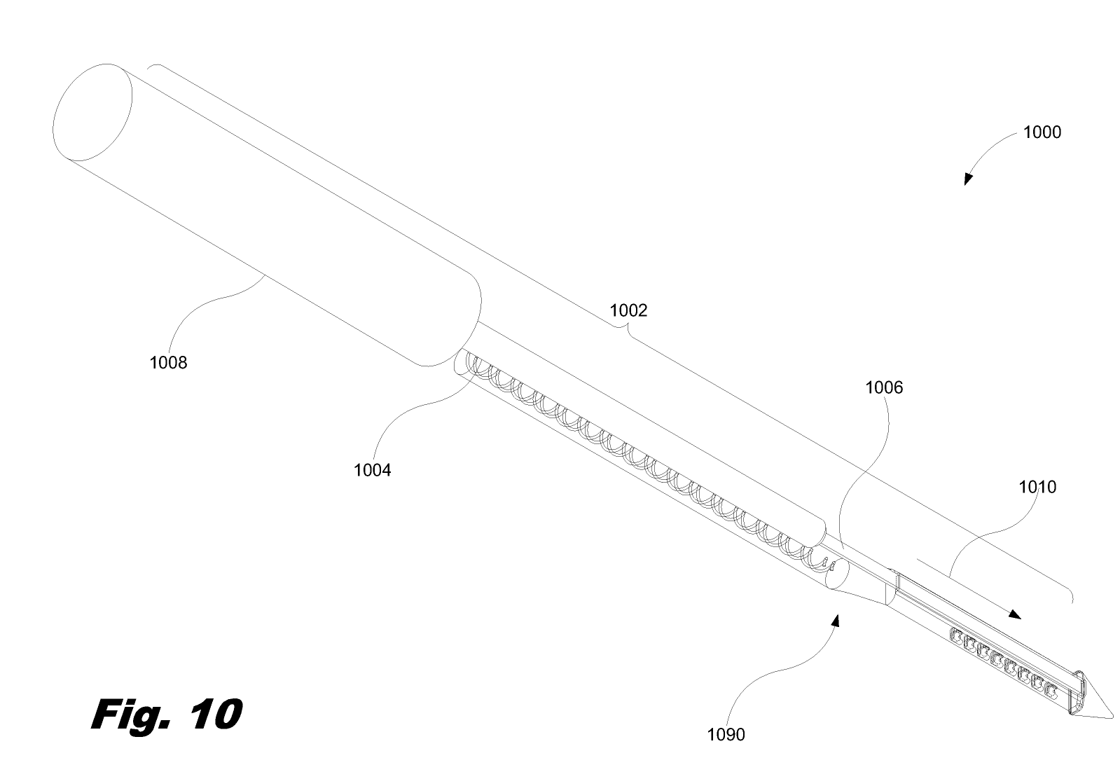

FIG. 10 is a perspective view of an intraneural electrode array implantation system (1000), according to another example of the principles described herein. The implantation system (1000) of FIG. 10 comprises an implantation tool (1002) and an intraneural electrode lead (1090) selectively coupled to the implantation tool (1002). The implantation tool (1002) comprises a main shaft (1004), a handle (1008) coupled to the main shaft, and a stylet (1006) onto which the intraneural electrode lead (1090) is coupled. The handle (1008) is used by, for example, a physician in steering the implantation system (1000) during implantation of the intraneural electrode lead (1090). The main shaft (1004), handle (1008), and stylet (1006) of the implantation tool (1002) are made of a generally rigid and durable material so that the implantation tool (1002) can enter bodily tissues without diverging from an intended insertion path or breaking due to stress. In one example, the implantation tool (1002) is made of stainless surgical steel.

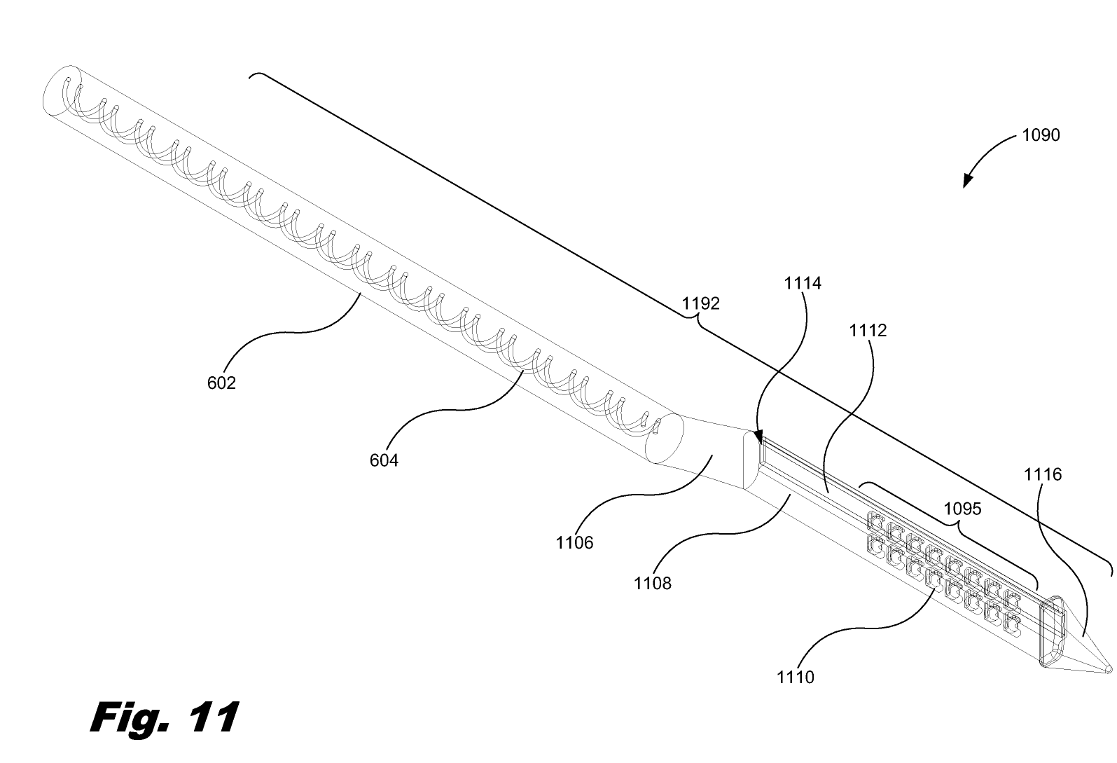

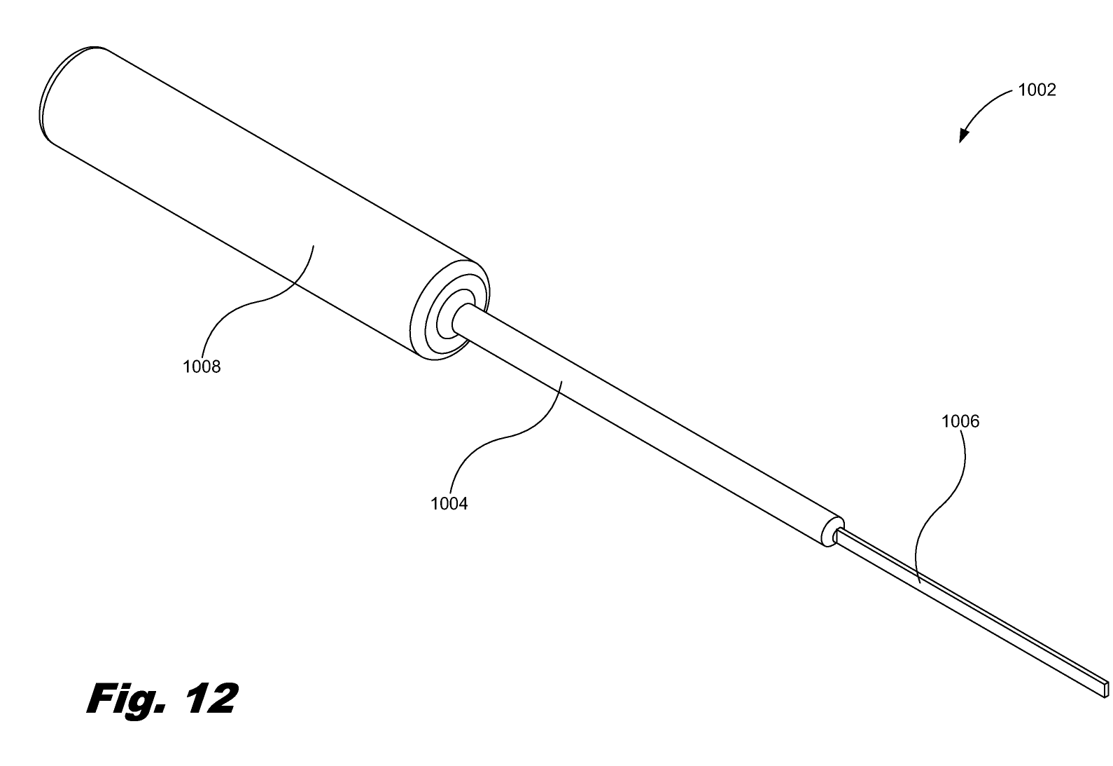

The stylet (1006) of the example of FIG. 10 is used as a portion to which the intraneural electrode lead (1090) can couple, and is also used to position the intraneural electrode lead (1090) within bodily tissues, and, more specifically, a cochlear nerve (160) as will now be described in more detail in connection with FIGS. 10, 11, and 12. FIG. 11 is a perspective view of the distal end of the intraneural electrode lead (1090) of the intraneural electrode array implantation system (1000) of FIG. 10, according to one example of the principles described herein. Further, FIG. 12 is a perspective view of the implantation tool (1002) of the intraneural electrode array implantation system (1000) of FIG. 10, according to one example of the principles described herein.

The stylet (1006) of the implantation system (1090) of FIGS. 10, 11, and 12 has a generally square cross section. Other cross-sectional shapes may be used for the stylet (1006), and a lumen (1114) with a matching cross-sectional shape is formed in the intraneural electrode lead (1090). The intraneural electrode lead (1090) depicted in FIGS. 10 and 11 comprises a lead body (1192) comprising a main lead portion (602) comprising a number of coiled electrode wires (604), a lead diverging arm (1106), and an electrode substrate portion (1108) comprising an intraneural electrode array (1095) comprising a number of electrodes (1110) and electrode wires traveling from the electrodes (1110) to the coiled electrode wires (604).

The intraneural electrode lead (1090) further comprises a sheath (1112) formed into a side of the intraneural electrode lead (1090) that defines a lumen (1114). The sheath (1112) and associated lumen (1114) are located on a side of the intraneural electrode lead (1090) to which the implantation tool (1002) can be coupled. Coupling of the intraneural electrode lead (1090) to the implantation tool (1002) is performed by bringing the stylet (1006) of the implantation tool (1002) in proximity to the lumen (1114) defined in the sheath (1112) of the intraneural electrode lead (1090). The stylet (1006) is inserted into the lumen (1114) in the direction of arrow (1010).

As described above, the stylet (1006) also serves to assist in the implantation and positioning of the intraneural electrode lead (1090) within bodily tissues. Once the intraneural electrode lead (1090) is coupled to the stylet (1006), the stylet is used to move the intraneural electrode lead (1090) through bodily tissues. In one example, the intraneural electrode lead (1090) further comprises a blunt dissector tip (1116). The blunt dissector tip (1116) comprises a tapering arrowhead-shape that is sharp enough to penetrate a number of types of bodily tissues, but blunt enough to separate and move past bodily tissues such as, for example, nerve tissues while eliminating or reducing the cutting of nerve cells.

In one example, the blunt dissector tip (1116) is made of a thermoplastic material such as, for example, polyether ether ketone (PEEK) or a polysulphone, or other polymers such as, for example, liquid-crystal polymers (LCP). The blunt dissector tip (1116) is embedded into the electrode substrate portion (1108) of the intraneural electrode lead (1090). In one example, during formation of the intraneural electrode lead (1090), a portion of the blunt dissector tip (1116) is integrated into the intraneural electrode lead (1090) by overmolding the blunt dissector tip (1116) along with, for example, the intraneural electrode array (1095) comprising the electrodes (1110), electrode wires, and coiled electrode wires (604).

Once inserted into the lumen (1114), the distal end of the stylet (1006) rests against a proximal edge of the blunt dissector tip (1116). In this manner, the stylet (1006) can be used to apply pressure to the blunt dissector tip (1116), move the intraneural electrode lead (1090) through bodily tissues, and implant the intraneural electrode lead (1090) into, for example, the cochlear nerve (160). In one example, the blunt dissector tip (1116) is larger in at least one dimension than the various portions of the intraneural electrode lead (1090) including, for example, the electrode substrate portion (1108), the lead diverging arm (1106), and main lead portion (602). For example, the blunt dissector tip (1116) has a larger maximum cross section on all sides than the cross section of any portion of the intraneural electrode lead (1090). In this manner, the blunt dissector tip (1116) can act to separate bodily tissues during implantation of the intraneural electrode lead (1090) without allowing the intraneural electrode lead (1090) to be displaced or disengaged from the implantation tool (1002). Further, once the intraneural electrode lead (1090) has been implanted into the desired bodily tissues, the blunt dissector tip (1116) in this example and other examples described herein acts as an anchor to retain the intraneural electrode lead (1090) in its originally implanted position within the bodily tissues.

Once the intraneural electrode lead (1090) is positioned within the intended bodily tissues (e.g., the cochlear nerve (160)), the implantation tool (1002) is pulled away from the intraneural electrode lead (1090), the stylet (1006) is removed from the lumen (1114) defined by the sheath (1112), and the implantation tool (1002) is removed from the bodily tissues.

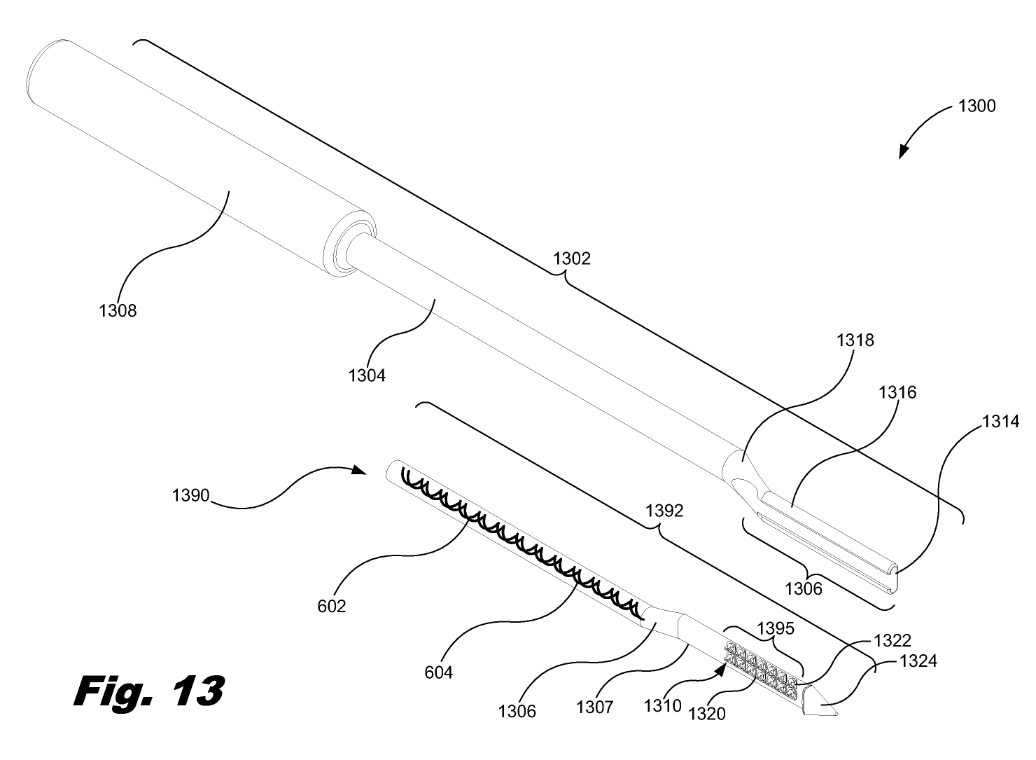

FIG. 13 is a perspective view of an intraneural electrode array implantation system (1300), according to still another example of the principles described herein. The implantation system (1300) comprises an implantation tool (1302) and an intraneural electrode lead (1390) that can be selectively coupled to the implantation tool (1302). The implantation tool (1302) comprises a main shaft (1304), a handle (1308) coupled to the main shaft (1304), and a lead receiving portion (1316). The handle (1308) is used by, for example, a physician in steering the implantation system (1300) during implantation of the intraneural electrode lead (1390). The main shaft (1304), handle (1308), and lead receiving portion (1316) of the implantation tool (1302) is made of a generally rigid and durable material so that the implantation tool (1302) can enter bodily tissues without diverging from an intended insertion path or breaking due to stress. In one example, the implantation tool (1302) is made of stainless surgical steel.

The implantation tool (1302) may further comprise a diverging arm (1318) that angularly extends from the main shaft (1304) to the lead receiving portion (1316). The diverging arm (1318) axially separates the main shaft (1304) from the lead receiving portion (1316). By axially separating the main shaft (1304) from the lead receiving portion (1316), the intraneural electrode lead (1390), when coupled to the implantation tool (1302), remains behind the lead receiving portion (1316) and below the main shaft (1304) and is not displaced or disengaged from the implantation tool (1302) when the implantation tool (1302) is inserted into bodily tissues.

In the example of FIG. 13, the lead receiving portion (1316) comprises a generally c-shaped cross section with respect to the longitudinal axis of the implantation tool (1302). In another example, the lead receiving portion (1316) may, instead, have a square, circular, elliptical, or polygonal cross section, and the intraneural electrode lead (1390) may have a matching cross section to couple with the implantation tool (1302) to create an interference fit between the lead receiving portion (1316) and the electrode substrate portion (1307) of the intraneural electrode lead (1390). The electrode substrate portion (1307) of the intraneural electrode lead (1390) is coupled to the lead receiving portion (1316) of the implantation tool (1302) by moving the electrode substrate portion (1307) into the recess defined by the lead receiving portion (1316).

The intraneural electrode lead (1390) depicted in FIG. 13 comprises a lead body (1392) comprising a main lead portion (602) comprising a number of coiled electrode wires (604), a lead diverging arm (1306), and an electrode substrate portion (1307) comprising an intraneural electrode array (1395) comprising a number of electrodes (1310) and electrode wires traveling from the electrodes (1310) to the coiled electrode wires (604). The lead diverging arm (1306) angularly extends from the main lead portion (602) to the electrode substrate portion (1307). In one example, the contour formed by the main lead portion (602), lead diverging arm (1306), and electrode substrate portion (1307) matches the contour formed by the main shaft (1304), diverging arm (1318), and lead receiving portion (1306).

The intraneural electrode array (1395) of FIG. 13 comprises any number of electrodes (1310) arranged in any manner along the electrode substrate portion (1307). The intraneural electrode array (1395) comprises a number of electrodes (1310) that will excite portions of the cochlear nerve (160). The electrodes (1310) each comprise an electrode body (1320) and a conductive pad (1322) as described above.

The lead receiving portion (1316) also serves to assist in the implantation and positioning of the intraneural electrode lead (1390) within bodily tissues. Once the intraneural electrode lead (1390) is coupled to the lead receiving portion (1316), the lead receiving portion (1316) is used to move the intraneural electrode lead (1390) through bodily tissues. In one example, the intraneural electrode lead (1390) further comprises a blunt dissector tip (1324). The blunt dissector tip (1324) comprises an arrowhead-shape that is sharp enough to penetrate a number of types of bodily tissues, but blunt enough to separate and move past bodily tissues such as, for example, nerve tissues while eliminating or reducing the cutting of nerve cells.

In one example, the blunt dissector tip (1324) is made of a thermoplastic material such as, for example, polyether ether ketone (PEEK) or a polysulphone, or other polymers such as, for example, liquid-crystal polymers (LCP). The blunt dissector tip (1324) is embedded into the electrode substrate portion (1307) of the intraneural electrode lead (1390). In one example, during formation of the intraneural electrode lead (1390), a portion of the blunt dissector tip (1324) is integrated into the intraneural electrode lead (1390) by overmolding the blunt dissector tip (1324) along with the intraneural electrode array (1395) comprising the electrodes (1310), electrode wires, and coiled electrode wires (604).

Once the intraneural electrode lead (1390) is coupled to the lead receiving portion (1316) of the implantation tool (1302), the distal end (1314) of the lead receiving portion (1316) rests against a proximal edge of the blunt dissector tip (1324). In this manner, the distal end (1314) of the lead receiving portion (1316) can be used to apply pressure to the blunt dissector tip (1324), move the intraneural electrode lead (1390) through bodily tissues, and implant the intraneural electrode lead (1390) into, for example, the cochlear nerve (160).

In one example, the blunt dissector tip (1324) is larger in at least one dimension than the various portions of the intraneural electrode lead (1390) and the implantation tool (1302) including, for example, the electrode substrate portion (1307), the lead diverging arm (1306), main lead portion (602), lead receiving portion (1316), diverging arm (1318), main shaft (1304), and combinations thereof. For example, the blunt dissector tip (1324) has a larger maximum cross section on all sides than the cross section of any portion of the intraneural electrode lead (1390), the implantation tool (1302), or combinations thereof. In this manner, the blunt dissector tip (1324) can act to separate bodily tissues during implantation of the intraneural electrode lead (1390) without allowing the intraneural electrode lead (1390) to be displaced or disengaged from the implantation tool (1302).

Once the intraneural electrode lead (1390) is positioned within the intended bodily tissues (e.g., the cochlear nerve (160)), the implantation tool (1302) is pulled away from the intraneural electrode lead (1390), the lead receiving portion (1316) is uncoupled from the electrode substrate portion (1307) of the intraneural electrode lead (1390), and the implantation tool (1302) is removed from the bodily tissues.

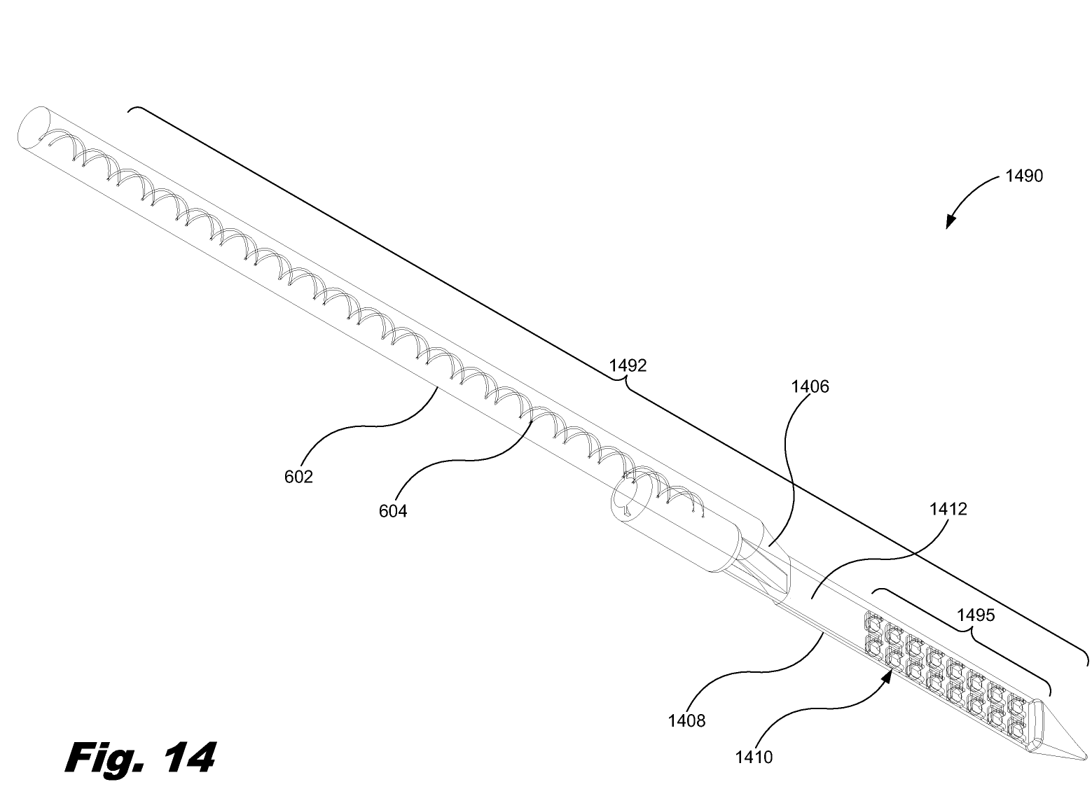

FIG. 14 is a perspective view of an intraneural electrode lead (1490), according to yet another example of the principles described herein. The intraneural electrode lead (1490) comprises a lead body (1492) comprising a main lead portion (602) comprising a number of coiled electrode wires (604), a lead diverging arm (1406), and an electrode substrate portion (1408) comprising an intraneural electrode array (1495) comprising a number of electrodes (1410) and electrode wires (1409). In the example of FIG. 14, the electrodes (1410) are coupled to a stiffener (1412) as will be discussed in more detail below. The stiffener (1412) reinforces the intraneural electrode lead (1490) during implantation of the intraneural electrode lead (1490) in bodily tissues.

In this example and other examples disclosed herein, the stiffener (1412) is made of a rigid material to reinforce the intraneural electrode array (1495) during implantation of the intraneural electrode lead (1490) in bodily tissues as described above. In one example, the stiffener is made of a polymer such as, for example, polyether ether ketone (PEEK), a polysulphone, or a liquid-crystal polymer (LCP).

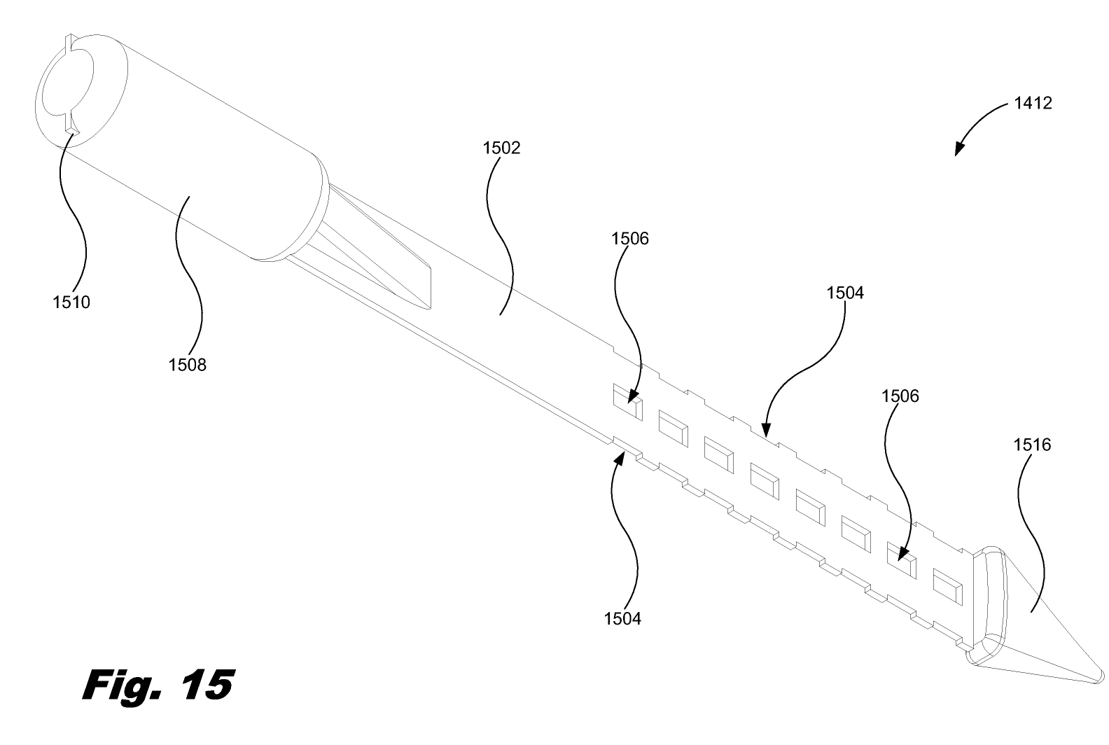

FIG. 15 is a perspective view of the stiffener (1412) of the intraneural electrode lead (1490) of FIG. 14, according to one example of the principles described herein. As depicted in FIGS. 14 and 15, the electrodes (1410) are coupled to the stiffener (1412) via a number of slots (1504) and voids (1506) defined in the body (1502) of the stiffener (1412). The slots (1504) and voids (1506) provide a portion of the body (1502) of the stiffener (1412) to which the electrodes (1410) can be coupled. In this manner, the electrodes (1410) are seated in the recesses defined by the slots (1504) and voids (1506), and are retained due to this recessed seating.

The stiffener (1412) further comprises a head (1508). The head (1508) is the portion of the stiffener (1412) to which force is applied to insert the intraneural electrode lead (1490). The head (1508) comprises a number of coupling elements (1510) to which a tool may be coupled to apply force to the head (1508). Still further, the stiffener (1412) comprises a blunt dissector tip (1516). The blunt dissector tip (1516) comprises a tapering arrowhead-shape that is sharp enough to penetrate a number of types of bodily tissues, but blunt enough to separate and move past bodily tissues such as, for example, nerve tissues while eliminating or reducing the cutting of nerve cells. In one example, during formation of the intraneural electrode lead (1490), a portion of the stiffener (1412) is integrated into the intraneural electrode lead (1490) by overmolding the stiffener (1412) along with, for example, the intraneural electrode array (1495) comprising the electrodes (1410), electrode wires, and coiled electrode wires (604). In one example, the blunt dissector tip (1516) and head (1508) are not overmolded, and the body (1502) is overmolded.

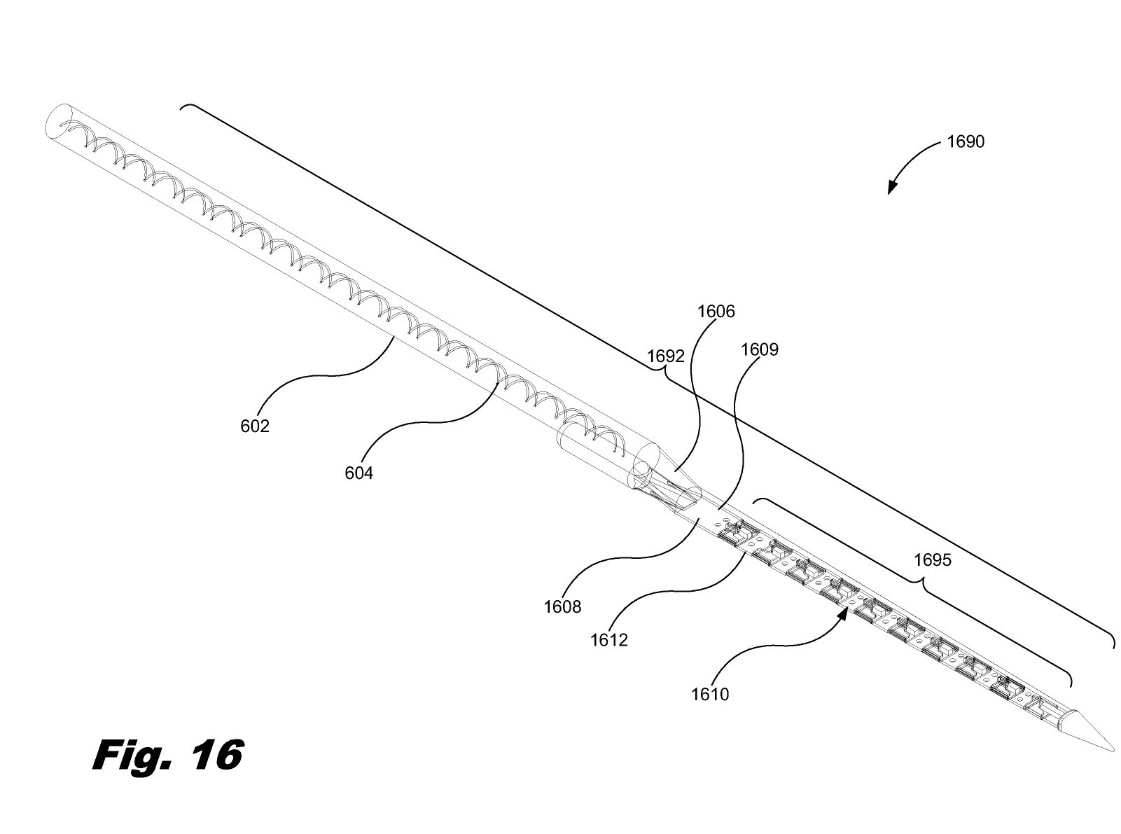

FIG. 16 is a perspective view of an intraneural electrode lead (1690), according to yet another example of the principles described herein. The intraneural electrode lead (1690) comprises a lead body (1692) comprising a main lead portion (602) comprising a number of coiled electrode wires (604), a lead diverging arm (1606), and an electrode substrate portion (1608) comprising an intraneural electrode array (1695) comprising a number of electrodes (1610) and electrode wires. In the example of FIG. 16, the electrodes (1610) are coupled to a stiffener (1612) as will be discussed in more detail below. The stiffener (1612) reinforces the intraneural electrode array (1495) during implantation of the intraneural electrode lead (1690) in bodily tissues.

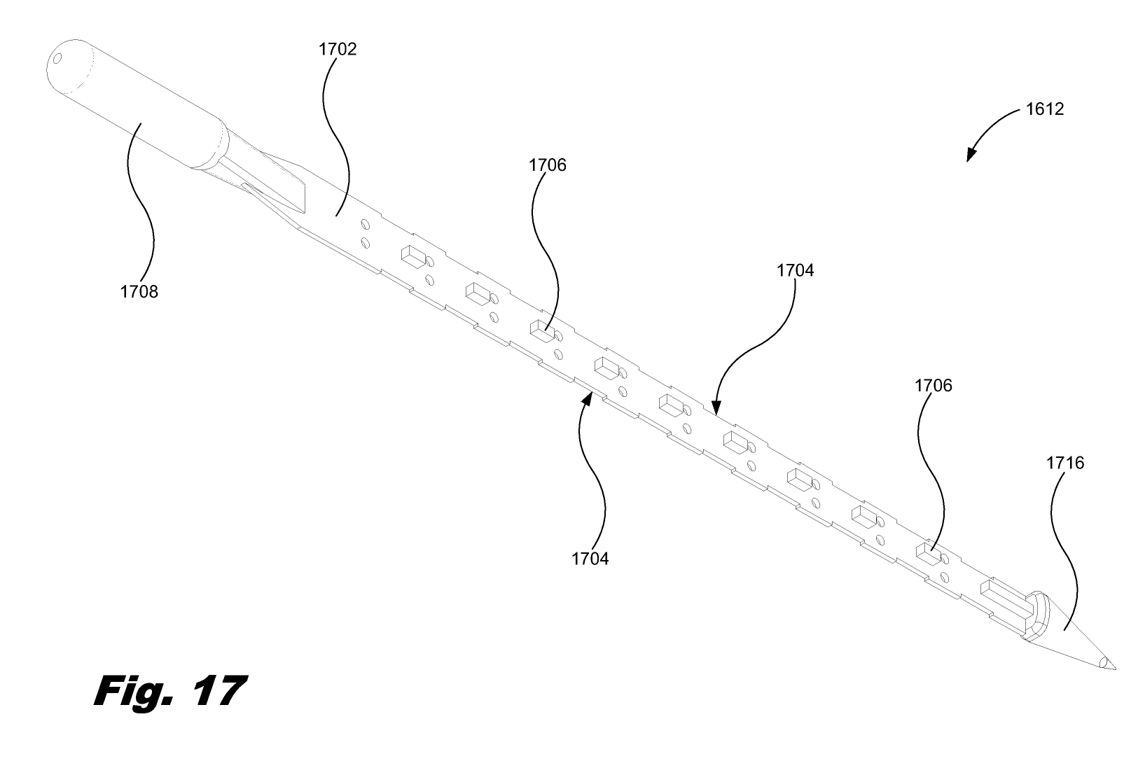

FIG. 17 is a perspective view of the stiffener (1612) of the intraneural electrode lead (1690) of FIG. 16, according to one example of the principles described herein. As depicted in FIGS. 16 and 17, the electrodes (1610) are coupled to the stiffener (1612) via a number of slots (1704) defined in the body (1702) of the stiffener (1612) and a number of protrusions (1706) formed in the body (1702) of the stiffener (1612). The slots (1704) and protrusions (1706) provide a portion of the body (1702) of the stiffener (1612) to which the electrodes (1610) can be coupled. In this manner, the electrodes (1610) are seated in the recesses defined by the slots (1704), and are retained due to this recessed seating and the protrusions (1706). The stiffener (1612) of FIGS. 16 and 17 accommodates a number of flat electrodes (1610) in a single inline row. However, any number of electrodes (1610) may be coupled to the stiffener (1612) of this example and other examples in any arrangement.

The stiffener (1612) further comprises a head (1708). The head (1708) is the portion of the stiffener (1612) to which force is applied to insert the intraneural electrode lead (1690). The stiffener (1612) further comprises a blunt dissector tip (1716). The blunt dissector tip (1716) comprises a tapering arrowhead-shape that is sharp enough to penetrate a number of types of bodily tissues, but blunt enough to separate and move past bodily tissues such as, for example, nerve tissues while eliminating or reducing the cutting of nerve cells. In one example, during formation of the intraneural electrode lead (1690), a portion of the stiffener (1612) is integrated into the intraneural electrode lead (1690) by overmolding the stiffener (1612) along with, for example, the intraneural electrode array (1695) comprising the electrodes (1610), electrode wires, and coiled electrode wires (604). In one example, the blunt dissector tip (1716) and head (1708) are not overmolded, and the body (1702) is overmolded.

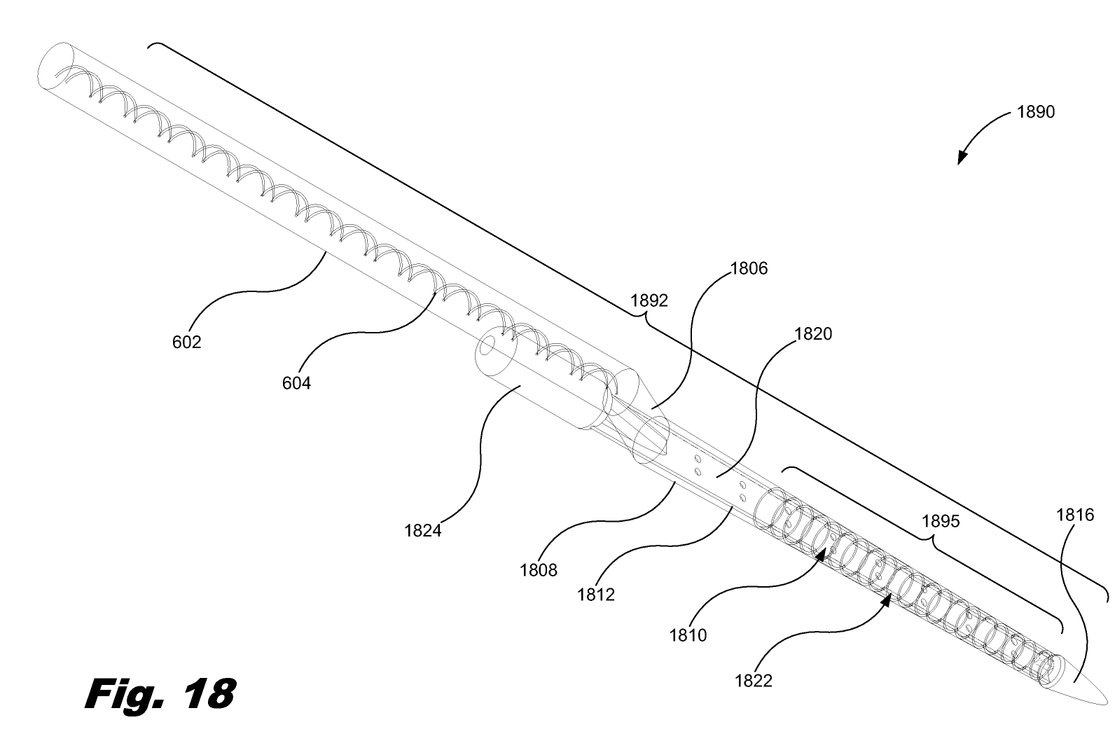

FIG. 18 is a perspective view of an intraneural lead (1890) comprising circular ring electrodes (1810), according to yet another example of the principles described herein. The intraneural lead (1890) comprises a lead body (1892) comprising a main lead portion (602) comprising a number of coiled electrode wires (604), a lead diverging arm (1806), and an electrode substrate portion (1808) comprising an intraneural electrode array (1895) comprising a number of electrodes (1810) and electrode wires. In the example of FIG. 18, the electrodes (1810) circumvent a stiffener (1812) as will be discussed in more detail below. The stiffener (1812) reinforces the intraneural lead (1890) during implantation of the intraneural lead (1890) in bodily tissues.

As depicted in FIG. 18, the electrodes (1810) are coupled to the stiffener (1812) via a number of slots (1822) defined in the body (1820) of the stiffener (1812). The slots (1822) provide a portion of the body (1820) of the stiffener (1812) to which the electrodes (1810) can be coupled. In this manner, the electrodes (1810) are seated in the recesses defined by the slots (1822). The stiffener (1812) of FIG. 18 accommodates a number of ring electrodes (1810) in a single inline row. However, any number of electrodes (1810) may be coupled to the stiffener (1812) of this example and other examples in any arrangement.

The stiffener (1812) further comprises a head (1824). The head (1824) is the portion of the stiffener (1812) to which force is applied to insert the intraneural lead (1890). The stiffener (1812) further comprises a blunt dissector tip (1816). The blunt dissector tip (1816) of FIG. 18 comprises a conical shape that is sharp enough to penetrate a number of types of bodily tissues, but blunt enough to separate and move past bodily tissues such as, for example, nerve tissues while eliminating or reducing the cutting of nerve cells. In one example, during formation of the intraneural lead (1890), a portion of the stiffener (1812) is integrated into the intraneural lead (1890) by overmolding the stiffener (1812) along with, for example, the intraneural electrode array (1895) comprising the electrodes (1810), electrode wires, and coiled electrode wires (604). In one example, the blunt dissector tip (1816) and head (1824) are not overmolded, and the body (1820) is overmolded.

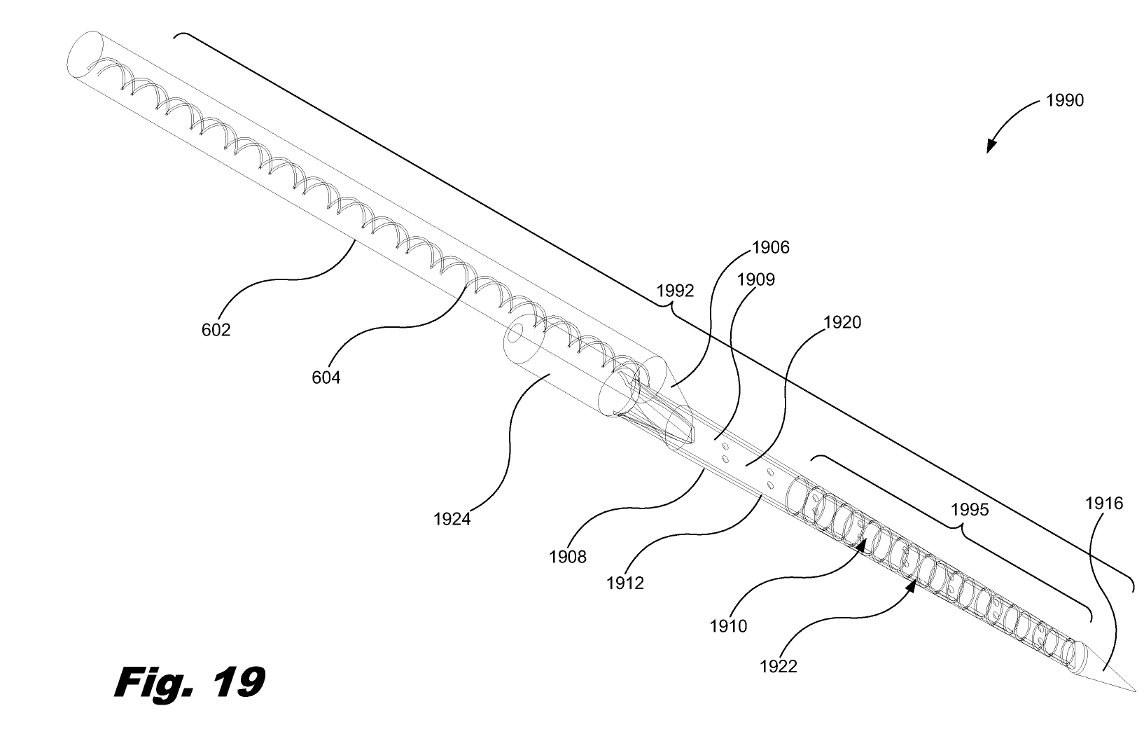

FIG. 19 is a perspective view of an intraneural lead (1990) comprising elliptical ring electrodes (1910), according to yet another example of the principles described herein. The intraneural lead (1990) comprises a lead body (1992) comprising a main lead portion (602) comprising a number of coiled electrode wires (604), a lead diverging arm (1906), and an electrode substrate portion (1908) comprising an intraneural electrode array (1995) comprising a number of electrodes (1910) and electrode wires (1909). In the example of FIG. 19, the electrodes (1910) circumvent a stiffener (1912) as will be discussed in more detail below. The stiffener (1912) reinforces the intraneural lead (1990) during implantation of the intraneural lead (1990) in bodily tissues.

As depicted in FIG. 19, the electrodes (1910) are coupled to the stiffener (1912) via a number of slots (1922) defined in the body (1920) of the stiffener (1912). The slots (1922) provide a portion of the body (1920) of the stiffener (1912) to which the electrodes (1910) can be formed around. In this manner, the electrodes (1910) are seated in the recesses defined by the slots (1922). The stiffener (1912) of FIG. 19 accommodates a number of elliptical ring electrodes (1910) in a single inline row. However, any number of electrodes (1910) may be coupled to the stiffener (1912) of this example and other examples in any arrangement.

The stiffener (1912) further comprises a head (1924). The head (1924) is the portion of the stiffener (1912) to which force is applied to insert the intraneural lead (1990). The stiffener (1912) also comprises a blunt dissector tip (1916). The blunt dissector tip (1916) of FIG. 19 comprises an elliptical conical shape that is sharp enough to penetrate a number of types of bodily tissues, but blunt enough to separate and move past bodily tissues such as, for example, nerve tissues while eliminating or reducing the cutting of nerve cells. In one example, during formation of the intraneural lead (1990), a portion of the stiffener (1912) is integrated into the intraneural lead (1990) by overmolding the stiffener (1912) along with, for example, the intraneural electrode array (1995) comprising the electrodes (1910), electrode wires, and coiled electrode wires (604). In one example, the blunt dissector tip (1916) and head (1924) are not overmolded, and the body (1920) is overmolded.

The specification and figures describe an intraneural implant. The intraneural implant comprises a lead comprising a number of electrode wires, a number of electrodes communicatively coupled to the electrode wires, the electrodes forming an electrode array, and an overmold surrounding the electrode wires and at least a portion of the electrodes, and a blunt dissector tip coupled to the lead to penetrate nerve tissues as the electrode array is implanted. An intraneural implant system comprises a flexible lead. The flexible lead comprises a lead body, an electrode array communicatively coupled to the lead body, a blunt dissector tip to penetrate a nerve bundle as the electrode array is implanted into the nerve bundle, and an implantation tool coupled to the electrode array to implant the electrode array into the nerve bundle.

The intraneural implant and implant system may have a number of advantages, including benefits of intraneural stimulation including frequency-specific stimulation across the entire cochlear frequency range, lower excitation thresholds, more restricted spread of excitation, reduced interference among simultaneously stimulated electrodes, and enhanced transmission of temporal fine structure. Further, the intraneural implant and implant system eliminate or reduce infection that may occur at the implantation site and assist in retention of the implanted intraneural electrode array in its originally implanted position within the cochlear nerve.

The preceding description has been presented to illustrate and describe examples of the principles described. This description is not intended to be exhaustive or to limit these principles to any precise form disclosed. Many modifications and variations are possible in light of the above teaching.

What is claimed is:

1. An intraneural implant comprising:
a lead comprising:
a number of electrode wires;
a number of electrodes communicatively coupled to the electrode wires, the electrodes forming an electrode array; and
an overmold surrounding the electrode wires and at least a portion of the electrodes; and
a blunt dissector tip coupled to the lead to penetrate nerve tissues as the electrode array is implanted.

2. The intraneural implant of claim 1, in which the electrode wires and electrodes comprise platinum or a platinum alloy.

3. The intraneural implant of claim 1, in which the overmold comprises a flexible material.

4. The intraneural implant of claim 3, in which the flexible material comprises silicone.

5. The intraneural implant of claim 1, in which the number of electrodes is between 10 and 20.

6. The intraneural implant of claim 1, in which the blunt dissector tip comprises a polyether ether ketone (PEEK), a polysulfone, or a liquid crystal polymer (LCP).

7. The intraneural implant of claim 1, further comprising a stiffener coupled to the electrode array to implant the electrode array.

8. The intraneural implant of claim 7, in which a portion of the stiffener is surrounded by the overmold.

9. The intraneural implant of claim 7, in which the electrodes are coupled to a portion of the stiffener.

10. The intraneural implant of claim 7, in which the stiffener comprises a polyether ether ketone (PEEK), a polysulfone, or a liquid crystal polymer (LCP).

11. The intraneural implant of claim 7, in which the stiffener comprises the blunt dissector tip.

12. The intraneural implant of claim 1, in which a portion of the blunt dissector tip is surrounded by the overmold.

13. The intraneural implant of claim 1, further comprising an implantation tool to implant the electrode array of the intraneural implant into a cochlear nerve.

14. The intraneural implant of claim 13, in which the implantation tool comprises the blunt dissector tip.

15. The intraneural implant of claim 13, further comprising:
a stylet coupled to the implantation tool; and
a lumen formed in the electrode array;
in which the electrode array is coupled to the implantation tool via the stylet.

16. The intraneural implant of claim 1, in which a portion of the longitudinal cross section of the blunt dissector tip is larger than the longitudinal cross section of the intraneural implant.

17. An intraneural implant system, comprising:
a flexible lead, comprising:
a lead body;
an electrode array communicatively coupled to the lead body;
a blunt dissector tip to penetrate a nerve bundle as the electrode array is implanted into the nerve bundle; and
an implantation tool coupled to the electrode array to implant the electrode array into the nerve bundle.

18. The intraneural implant system of claim 17, further comprising a stiffener coupled to the blunt dissector tip to carry the electrode array through the nerve bundle during implantation.

19. The intraneural implant system of claim 18, in which the stiffener is decoupled from the blunt dissector tip after implantation of the electrode array in the nerve bundle.

20. The intraneural implant system of claim 18, in which the stiffener is decoupled from the electrode array after implantation of the electrode array in the nerve bundle.

21. The intraneural implant system of claim 18, in which the stiffener is implanted with the electrode array.

22. The intraneural implant system of claim 17, further comprising a locking ring to lockingly engage the electrode array to the implantation tool.

23. The intraneural implant system of claim 17, in which the electrode array comprises a number of electrodes, and in which each electrode comprises a ring electrode.

24. An intraneural implant tool comprising:
a stylet,
in which the stylet retains an intraneural lead and applies force to a portion of the intraneural lead during implantation of an electrode array coupled to the intraneural lead in a nerve bundle.

25. An intraneural implant tool comprising:
a lead receiving portion comprising a blunt dissector tip,
in which the lead receiving portion retains an intraneural lead, and
in which the blunt dissector tip enters a nerve bundle during implantation of an electrode array coupled to the intraneural lead in the nerve bundle.
